(19) United States
(12) Patent Application Publication
(10) Pub. No.: US 2004/0244794 A1
(43) Pub. Date: Dec. 9, 2004

(54) Inhalation device with a pharmaceutical composition

(76) Inventor: David Hugh Richards, Greenford (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/486,021

(22) PCT Filed: Aug. 5, 2002
(86) PCT No.: PCT/EP02/08718

(30) Foreign Application Priority Data
Aug. 9, 2001 (GB) 0119411.7
Aug. 9, 2001 (GB) 0119408.3

(51) Int. Cl. A61M 15/00

(52) U.S. Cl. 128/203.15; 128/203.12

(57) ABSTRACT

According to the present invention there is provided an inhalation device comprising plural doses of medicament in powder form, wherein the medicament is a pharmaceutical formulation comprising salmeterol or a pharmaceutically acceptable salt, solvate, or physiologically functional derivative thereof and an anticholinergic agent or a pharmaceutically acceptable salt, solvate, or physiologically functional derivative thereof, and a pharmaceutically acceptable carrier or excipient, and optionally one or more other therapeutic ingredients.

### TECHNICAL FIELD

The present invention relates to an inhalation device comprising a medicament pack containing an inhalable medicament in powder form, the medicament being a pharmaceutical formulation comprising salmeterol and an anticholinergic agent for the treatment of respiratory diseases.

### BACKGROUND TO THE INVENTION

Inhalation devices are known for use with blister packs in which the medicament is held in powder form in the blisters thereof. These devices can be used to administer medicaments for the treatment of respiratory disorders associated with reversible airway obstruction such as asthma, chronic obstructive pulmonary disease (COPD), respiratory tract infection and upper respiratory tract disease.

A number of bronchodilatory medicaments are known in the art for the treatment of asthma and related disorders. Thus, for example, GB 2 140 800 describes phenethanolamine compounds which are β2-adrenoreceptor agonists including 4-hydroxy-α1-[[[6-(4-phenylbutoxy)hexyl]-amino]methyl]-1,3-benzenedimethanol 1-hydroxy-2-naphthalenecarboxylate (salmeterol xinafoate) which is now used clinically in the treatment of bronchial asthma and related disorders.

Similarly, U.S. Pat. No. 3,505,337 and U.S. Pat. No. 3,681,500 describe ipratropium and its salts, such (endo,syn)-(+/−)-3-(3-hydroxy-1-oxo-2-phenylpropoxy)-8-methyl-8-(1-methylethyl)-8-azoniabicyclo[3.2.1]octane bromide (ipratropium bromide) and pharmaceutical formulations thereof. Ipratropium bromide is an anticholinergic agent, which is now used clinically in the treatment of bronchial asthma and related disorders.

Atropine (1αH,5 αH-Tropan-3α-ol (+/−)-tropate ester-), oxitropium bromide ((8r)-6 β,7β-epoxy-8-ethyl-3α-hydroxy-1αH, 5αH-tropanium bromide (−)-tropate), tiotropium bromide (6β,7β-epoxy-3β-hydroxy-8-methyl-1αH, 5αH-tropanium bromide, di-2-thienylglycolate) and revatropate ((R)-3-quinuclidyl-(2)-2-hydroxymethyl-4-(R)-methylsulfinyl-2-phenyl butryate) are known anticholinergic agents. These medicaments are used clinically in the treatment of bronchial asthma and related disorders.

Although salmeterol xinafoate and the above anticholinergic drugs are effective bronchodilators, the maximum duration of action of the former is 12 hours whilst that of the anticholinergic drugs can be as little as 6 hours. For this reason, twice to three/four times daily dosing regimes, respectfully, can be required for these drugs. There is therefore a clinical need for bronchodilators having potent and selective action and having an advantageous profile of action.

It is an object of the present invention to provide inhalation devices containing medicament compositions which are effective at treating bronchial asthma and related disorders in powder form which may be administered by a multi-dose inhalation device.

Patients may also forget to take drugs at prescribed intervals, particularly the elderly or confused. Thus any medicaments which reduce or simplify a patient's treatment regime are desirable since they are more likely to be regularly taken.

It is a therefore a further object of the present invention to provide an inhalation device which will aid patient compliance by reducing the number of daily administrations of bronchodilatory drugs.

### SUMMARY OF THE INVENTION

According to the present invention there is provided an inhalation device comprising plural doses of medicament in powder form, wherein the medicament is a pharmaceutical formulation comprising salmeterol or a pharmaceutically acceptable salt, solvate, or physiologically functional derivative thereof and an anticholinergic agent or a pharmaceutically acceptable salt, solvate, or physiologically functional derivative thereof, and a pharmaceutically acceptable carrier or excipient, and optionally one or more other therapeutic ingredients.

In one aspect, the pharmaceutical formulation comprises salmeterol xinafoate and the anticholinergic agent, and a pharmaceutically acceptable carrier or excipient, and optionally one or more other therapeutic ingredients.

Suitably the anticholinergic agent is selected from the group consisting of ipratropium bromide, oxitropium bromide, tiotropium bromide, atropine sulfate, revatropate and any mixtures thereof.

In another aspect, the pharmaceutical formulation is suitable for administration by inhalation. Preferably, the pharmaceutical formulation is a dry powder. More preferably, the pharmaceutically acceptable carrier or excipient is lactose.

It is to be understood that the present invention covers all combinations of particular and preferred aspects of the invention described herein.

As would be appreciated by the skilled person, salmeterol and the anticholinergic agents ipratropium, atropine, tiotropium, oxitropium and revatropate all include at least one asymetric centre. The present invention includes both (S) and (R) enantiomers of salmeterol and the anticholinergic agents either in substantially pure form or admixed in any proportions. The enantiomers of salmeterol have been described previously, for example, in EP0422889 and WO 99/13867.

By the term “physiologically functional derivative” is meant a chemical derivative of salmeterol or the anticholinergic agents having the same physiological function as the free compound, for example, by being convertible in the body thereto. According to the present invention, examples of physiologically functional derivatives include esters.

Suitable salts according to the invention include those formed with both organic and inorganic acids. Pharmaceutically acceptable acid addition salts include but are not limited to those formed from hydrochloric, hydrobromic, sulphuric, citric, tartaric, phosphoric, lactic, pyruvic, acetic, trifluoroacetic, succinic, oxalic, fumaric, maleic, oxaloacetic, methanesulphonic, ethanesulphonic, p-toluenesulphonic, benzenesulphonic, isethionic, and naphthalenecarboxylic, such as 1-hydroxy-2-naphthalenecarboxylic acids.

Pharmaceutically acceptable esters of salmeterol or the anticholinergic agents atropine, oxitropium, tiotropiium and revatropate may have a hydroxyl group converted to a C1-6alkyl, aryl, aryl C1-6 alkyl, or amino acid ester.

In a further aspect, the medicament is contained on a retainer comprising a mesh disc or velour disc. Preferably the medicament is contained within a multi-dose blister pack.

In one aspect, at least one container for the medicament in the multi-dose blister pack is defined between two members peelably secured to one another, the device comprising means defining an opening station for the at least one container; means for peeling the members apart at the opening station to open the container; and an outlet, communicating with the opened container, through which a user can inhale medicament in powder form from the opened container.

In another aspect, the inhalation device is adapted for use where the two members are two sheets. Preferably, the device is adapted for use where the sheets are elongate sheets which define a plurality of medicament containers spaced along the length thereof, the device being provided with indexing means for indexing each container in turn in communication with the outlet. More preferably, the device is adapted for use where one of the sheets is a base sheet having a plurality of pockets therein, and the other of the sheets is a lid sheet, each pocket and the adjacent part of the lid sheet defining a respective one of the containers, the device comprising driving means for pulling the lid sheet and base sheet apart at the opening station.

In another aspect, the driving means comprises lid driving means for pulling the lid sheet.

In another aspect, the device comprises a rotatable index wheel having recesses therein, the wheel being engageable with the medicament pack so that the recesses each receive a respective pocket.

Preferably, the index wheel and the lid driving means are interconnected so that the rotation of one correlates with the rotation of the other. More preferably, the index wheel and lid driving means are interconnected by a slipping clutch. More preferably, the slipping clutch comprises a first gear member which is movable with the index wheel and has a toothed surface, and a second toothed gear member which is movable with the lid driving means and has a toothed surface in meshing engagement with the toothed surface of the said first gear member, at least one of the toothed surfaces having a toothed portion which is movable back and forth with respect to the remainder of the toothed surface of which it is part.

In a further aspect, the slipping clutch comprises first clutch means movable with the index wheel and second clutch means movable with the lid driving means, one of the clutch means comprising an annular array of serrations and the other of the clutch means comprises means which grippingly engage the serrations when less than a predetermined force is applied between the two clutch means and which slip with respect to the serrations when a force equal to or greater than the predetermined force is applied.

Preferably, the lid driving means comprises a wheel on which the lid sheet is wound up, the wheel having a winding surface which decreases in diameter when tension in the lid sheet increases.

Preferably, the wheel comprises a plurality of resiliently flexible arms each extending therefrom at an angle with respect to a radius.

In one aspect, the inhalation device comprises indexing means engageable between adjacent pockets to cause each pocket in turn to be positioned in communication with the outlet.

Preferably, the lid driving means comprises a pair of driving wheels which drivingly engage the lid sheet between them. More preferably, the driving wheels are toothed wheels having interengaging teeth.

In another aspect, the inhalation device comprises means for guiding the lid sheet and base sheet along separate paths at the opening station, the paths reuniting downstream of the opening station, the driving means being located after the point where the paths reunite and being operable to drive both the lid sheet and base sheet. Preferably, the driving means comprises a pair of toothed wheels having interengaging teeth.

In a further aspect, the inhalation device comprises at least one chamber for receiving the elongate medicament pack before opening, and for receiving the base sheet and lid sheet after peeling apart.

Preferably, the elongate medicament pack and/or base sheet are held in coiled form by resilient coil-formers.

More preferably, the inhalation device is operable in a plurality of steps, which comprises indicator means adapted to display to a user an instruction as to the next step once the preceding step has been taken.

Preferably, the indicator means comprise an indicator member which carries a plurality of legends each constituting an instruction to the user, the indicator member being movable by a given step being carried out to display the legend relating to the next step.

In one aspect, the inhalation device comprises a housing with a cylindrical chamber therein; an air inlet into the chamber; a support inside the chamber arranged to support the blister pack comprising a plurality of containers arranged in a circle; a plunger operable to engage a container registered therewith to open the container is such a way that air being inhaled by a patient will cause the medicament to be released therefrom; means for rotating the blister pack on the support to register each container in turn with the plunger; and, communicating with the interior of the chamber, a mouthpiece outlet through which a patient can inhale whereby medicament will be released from a container and entrained in the airflow produced by the patient so as to pass through the outlet.

Preferably, the support is a rotatable plate with a plurality of holes therethrough, the holes being arranged in a circle and each being adapted to receive a container for medicament, a rotatable member being located outside the chamber and connected with the support so that rotation of the said member will cause rotation of the support; a mouthpiece outlet leading from the chamber in a substantially radial direction; and a perforated guard positioned so that air and medicament inhaled through the mouthpiece will first pass through said guard.

In another aspect, the support is a rim inside the chamber and a clamp member is fitted inside the chamber and on the support but is removable to permit the blister pack to be placed on the support and thereafter clamped between the clamp member and the support, the clamp member having a plurality of holes arranged in a circle to receive a plurality of containers, being rotatable and arranged to rotate with it the blister pack clamped between the clamp member and the support, wherein an external knob is provided to rotate the clamp member and an outlet mouthpiece leads substantially radially from the chamber.

Preferably, the chamber has a cover which is removable to permit the blister pack to be inserted in the chamber and placed on the support, the plunger being carried by the cover.

More preferably, the mouthpiece is enclosed in a removable mouthpiece cover, the mouthpiece cover having means for preventing operation of the plunger when the mouthpiece cover is fitted on the mouthpiece.

In a further aspect, the blister pack is a circular disc having a plurality of frangible containers arranged in a circle and containing medicament in powder form.

In one aspect, the device comprises a body defining a reservoir for the medicament, an outlet through which a user can inhale, and a dosing member with at least one metering means formed therein, the dosing member being movable between a first position in which the at least one metering means communicates with the reservoir to receive a dose of medicament therefrom and a second position in which the at least one metering means communicates with the outlet to permit the user to inhale the dose.

In another aspect, the at least one metering means is formed in a face of the dosing member, the face being urged into contact against a similar mating face of the body at the lower end of the reservoir to form a dynamic seal.

Preferably, the flexible material has a coefficient of friction of 0.4 or less.

In one aspect, the faces are flat. More preferably, the mating face of the body is made of a flexible material. More preferably, the mating face of the body comprises a rubber insert having a hardness of between 40 and 60 Shore A.

In another aspect, the rubber insert comprises chlorinated butyl or butyl laminated with a contacting face made of a layer of PTFE, polypropylene or polyethylene. Preferably, the face of the dosing member is of unitary construction with the dosing member.

In one aspect, the dosing member is rotatably movable with respect to the reservoir about a common central axis such that the metering means are selectively in communication with the reservoir to receive the dose of medicament therefrom or with the outlet to release the dose of medicament to the user.

In another aspect, the device additionally comprises a reversibly removable reservoir cap to cover the reservoir and automatically load the metering means for use on removal of the cap.

In a further aspect, the device additionally comprises loading means for transferring medicament from the reservoir into the metering means.

Preferably, the loading means comprises a resilient scraper means positioned to first push medicament powder into the metering means and then remove excess medicament powder from the face of the dosing member to ensure that each metering means contains a reproducible amount of medicament powder.

More preferably, each metering means is in the form of a perforation or cavity for receipt of a pre-determined volume of medicament powder therein.

More preferably, the metering member is a flat plate.

In one aspect, the device additionally comprises counter means for providing a visual count of the number of doses of powdered medicament used or remaining within the reservoir.

Preferably, the device is actuable in response to the breath of a user.

In another aspect of the present invention, there is provided a method for the prophylaxis or treatment of a clinical condition in a mammal, such as a human, for which a selective β2-adrenoreceptor agonist and/or anticholinergic agent is indicated, which comprises administration of a therapeutically effective amount of a pharmaceutical formulation comprising salmeterol or a pharmaceutically acceptable salt, solvate, or physiologically functional derivative thereof and the anticholinergic agent or a pharmaceutically acceptable salt, solvate, or physiologically functional derivative thereof, and a pharmaceutically acceptable carrier or excipient, and optionally one or more other therapeutic ingredients, using the inhalation device of the present invention.

In a preferred aspect, there is provided a method which comprises administration of a therapeutically effective amount of a pharmaceutical formulation comprising salmeterol xinafoate and an anticholinergic agent and a pharmaceutically acceptable carrier or excipient.

Suitably the anticholinergic agent is selected from the group consisting of ipratropium bromide, oxitropium bromide, tiotropium bromide, atropine sulfate, revatropate and any mixtures thereof.

In particular, the present invention provides such a method for the prophylaxis or treatment of a disease associated with reversible airways obstruction such as asthma, chronic obstructive pulmonary disease (COPD), respiratory tract infection or upper respiratory tract disease.

The amount of salmeterol and anticholinergic agent, or a pharmaceutically acceptable salt, solvate or physiologically functional derivative thereof which is required to achieve a therapeutic effect will, of course, vary with the particular compound, the route of administration, the subject under treatment, and the particular disorder or disease being treated. As a monotherapy, salmeterol xinafoate is generally administered to adult humans by aerosol inhalation at a dose of 50 mcg or 100 mcg twice daily. The dosage requirements of anticholinergic agents vary; thus for example, as a monotherapy, tiotropium and oxitropium are administered to adult humans by inhalation at a dose of from 18 mcg to 200 mcg from one to three times daily.

Hereinafter, the term “active ingredients” means salmeterol or a pharmaceutically acceptable salt, solvate, or physiologically functional derivative thereof, preferably salmeterol xinafoate or a pharmaceutically acceptable salt, solvate, or physiologically functional derivative thereof, together with a pharmaceutically acceptable salt, solvate or physiologically active derivative of a anticholinergic agent, such as ipratropium, atropine, oxitropium, tiotropium or revatropate and amy mixtures thereof.

Suitably, the pharmaceutical formulations which are suitable for inhalation according to the invention comprise the active ingredients in amounts such that each actuation provides a therapeutically effective dose, for example, a dose of salmeterol of 10 mcg to 150 mcg, preferably 50 mcg and a dose of oxitropium bromide of 10 mcg to 400 mcg, preferably 200 mcg. The pharmaceutical formulations according to the invention may further include other therapeutic agents for example anti-inflammatory agents such as corticosteroids or other β2-adrenoreceptor agonists (such as salbutamol, formoterol, fenoterol or terbutaline and salts thereof).

Suitably, the pharmaceutical formulations which are suitable for inhalation according to the invention provide therapeutically effective doses that permit the establishment of a twice daily (bis in diem—b.i.d) dosing regimen.

The formulations may conveniently be presented in unit dosage form and may be prepared by any of the methods well known in the art of pharmacy. All methods include the step of bringing the active ingredients into association with the carrier which constitutes one or more accessory ingredients. In general the formulations are prepared by uniformly and intimately bringing into association the active ingredients with finely divided solid carriers and then, if necessary, shaping the product into the desired formulation. Formulations for inhalation include powder compositions which will preferably contain lactose. Blisters of, for example, laminated aluminium foil, for use in an inhaler may be formulated containing a powder mix of the active ingredients and a suitable powder base such as lactose or starch, preferably lactose. In this aspect, the active ingredients are suitably micronised so as to permit inhalation of substantially all of the active ingredients into the lungs upon administration of the dry powder formulation, thus the active ingredients will have a particle size of less than 100 microns, desirably less than 20 microns, and preferably in the range 1 to 10 microns.

Preferred unit dosage formulations are those containing a pharmaceutically effective dose, as hereinbefore recited, or an appropriate fraction thereof, of the active ingredient.

It should be understood that in addition to the ingredients particularly mentioned above, the formulations of this invention may include other agents conventional in the art having regard to the type of formulation in question. Furthermore, the claimed formulations include bioequivalents as defined by the US Food and Drugs Agency.

For a better understanding of the invention, the following Examples are given by way of illustration using oxitropium bromide and ipratropium bromided as typical examples of anticholinergic agents.

### EXAMPLES

EXAMPLE 1
50/80 salmeterol/oxitropium

| | Per blister | |
|---|---|---|
| Salmeterol Xinafoate | 72.6 | microgram |
| Oxitropium Bromide | 76.7 | microgram |
| Lactose Ph. Eur. | to 12.5 | mg |
| | or to 25.0 | mg |

The active ingredients are micronised and bulk blended with the lactose in the proportions given above. The blend is filled into specifically constructed double foil blister packs to be administered by an inhaler according to the present invention.

A similar method may be used for the formulations of Examples 2 to 6:

Per blister

Example 2: 50/120 salmeterol/oxitropium

| | | |
|---|---|---|
| Salmeterol Xinafoate | 72.6 | microgram |
| Oxitropium Bromide | 115 | microgram |
| Lactose Ph. Eur. | to 12.5 | mg |
| | or to 25.0 | mg |

Example 3: 50/160 salmeterol/oxitropium

| | | |
|---|---|---|
| Salmeterol Xinafoate | 72.6 | microgram |
| Oxitropium Bromide | 153.3 | microgram |
| Lactose Ph. Eur. | to 12.5 | mg |
| | or to 25.0 | mg |

Example 4: 50/320 salmeterol/oxitropium

| | | |
|---|---|---|
| Salmeterol Xinafoate | 72.6 | microgram |
| Oxitropium Bromide | 306.6 | microgram |
| Lactose Ph. Eur. | to 12.5 | mg |
| | or to 25.0 | mg |

Example 5: 50/80 salmeterol/oxitropium

| | | |
|---|---|---|
| Salmeterol Xinafoate | 72.5 | microgram |
| Oxitropium Bromide | 76.7 | microgram |
| Lactose Ph. Eur. | to 12.5 | mg |

The active ingredients were micronised and bulk blended with the lactose in the proportions given above (total blend size 4 kg). The blend was filled into specifically constructed double foil blister packs to be administered by the inhalation device of the present invention.

A similar method was used for the formulation of Example 6. The total blend size was 4 kg.

Per blister

Example 6: 50/160 salmeterol/oxitropium

| | | |
|---|---|---|
| Salmeterol Xinafoate | 72.5 | microgram |
| Oxitropium Bromide | 153.3 | microgram |
| Lactose Ph. Eur. | to 12.5 | mg |

Example 7: 50/80 salmeterol/ipratropium

| | | |
|---|---|---|
| Salmeterol Xinafoate | 72.6 | microgram |
| Ipratropium Bromide | 80 | microgram |
| Lactose Ph. Eur. | to 12.5 | mg |
| | or to 25.0 | mg |

The active ingredients are micronised and bulk blended with the lactose in the proportions given above. The blend is filled into specifically constructed double foil blister packs to be administered by an inhaler according to the present invention.

A similar method may be used for the formulations of Examples 8 to 12:

Per blister

Example 8: 50/120 salmeterol/ipratropium

| | | |
|---|---|---|
| Salmeterol Xinafoate | 72.6 | microgram |
| Ipratropium Bromide | 120 | microgram |
| Lactose Ph. Eur. | to 12.5 | mg |
| | or to 25.0 | mg |

Example 9: 50/160 salmeterol/ipratropium

| | | |
|---|---|---|
| Salmeterol Xinafoate | 72.6 | microgram |
| Ipratropium Bromide | 160 | microgram |
| Lactose Ph. Eur. | to 12.5 | mg |
| | or to 25.0 | mg |

Example 10: 50/320 salmeterol/ipratropium

| | | |
|---|---|---|
| Salmeterol Xinafoate | 72.6 | microgram |
| Ipratropium Bromide | 320 | microgram |
| Lactose Ph. Eur. | to 12.5 | mg |
| | or to 25.0 | mg |

Example 11: 50/80 salmeterol/ipratropium

| | | |
|---|---|---|
| Salmeterol Xinafoate | 72.5 | microgram |
| Ipratropium Bromide | 80 | microgram |
| Lactose Ph. Eur. | to 12.5 | mg |

The active ingredients were micronised and bulk blended with the lactose in the proportions given above (total blend size 4 kg). The blend was filled into specifically constructed double foil blister packs to be administered by the inhalation device of the present invention.

A similar method was used for the formulation of Example 12. The total blend size was 4 kg.

EXAMPLE 12
50/160 salmeterol/ipratropium

| | Per blister | |
|---|---|---|
| Salmeterol Xinafoate | 72.5 | microgram |
| Ipratropium Bromide | 160 | microgram |
| Lactose Ph. Eur. | to 12.5 | mg |

### BRIEF DESCRIPTION OF THE DRAWINGS

Further characteristics of the present invention will become apparent from the following description and accompanying drawings, wherein:

FIG. 1 is a rear view of a first embodiment of the invention;

FIG. 2 is an axonometric exploded view of the components of the embodiment of FIG. 1;

FIGS. 3a, 3b and 3c are an axonometric view, a longitudinal section and an end view (partly broken away) showing a clutch used in the embodiment of FIGS. 1 and 2;

FIGS. 4a and 4b are an axial section and cross-section respectively, on a larger scale than FIGS. 1 and 2, of a mouthpiece which may be used in the first embodiment (or in some other embodiment);

FIG. 5 is a front view of a second embodiment, with a cover thereof removed to show the interior;

FIG. 6 is a rear view of the second embodiment, but showing the interior thereof

FIG. 7 is an axonometric front view of the second embodiment;

FIG. 8 is an axonometric rear view of the second embodiment;

FIG. 9 is an axonometric exploded view of the second embodiment;

FIG. 10 is a front view of a third embodiment, showing the interior structure thereof;

FIG. 11 is an axial view, on a larger scale, showing the mouthpiece of the third embodiment;

FIG. 12 is a view from below of the third embodiment;

FIGS. 13 to 16 show a fourth embodiment of the invention, FIG. 13 being an underplan view, FIG. 14 a section on line A-A in FIG. 13, FIG. 15 a section on line B-B in FIG. 13, and FIG. 16 an exploded view on a smaller scale;

FIGS. 16a to 16d show the fourth embodiment in successive stages of operation, and FIG. 16e is a section taken on line A-A in FIG. 16a;

FIGS. 17 to 20 show a fifth embodiment of the invention, FIG. 17 being an end view, FIG. 18 a section on line A-A in FIG. 17, FIG. 19 a section on line B-B in FIG. 17, and FIG. 20 an exploded view;

FIGS. 21 to 24 show a sixth embodiment of the invention, FIG. 21 being an end view, FIG. 22 a section on line A-A in FIG. 21, FIG. 23 a section on line B-B in FIG. 21, and FIG. 24 an exploded view;

FIGS. 25 to 29 show a modified clutch which may be used in those embodiments of the invention which require it, and are, respectively, a front view, a top view, a back view, a left side view and an axonometric view;

FIG. 30 is an exploded perspective view showing a further embodiment of clutch which may be used;

FIG. 31 is an exploded perspective view of yet another embodiment of clutch which may be used;

FIG. 31a is transverse section through the clutch shown in FIG. 31;

FIGS. 32 to 34 show successive positions of operation of a seventh embodiment of the invention, in rear view;

FIG. 35 is a perspective view on a larger scale showing an embodiment of medicament pack according to the invention;

FIG. 36 is an exploded perspective view of a device according to an eighth embodiment of the invention.

FIG. 37 is a detailed view of a plunger device illustrated in FIG. 36;

FIG. 38 is an elevation of a ninth embodiment of the invention;

FIG. 39 is an exploded view of the embodiment illustrated in FIG. 38;

FIG. 40 is a perspective view of a tenth embodiment of the invention;

FIG. 41 is a cross-sectional view of an eleventh embodiment of the invention;

FIG. 42 is a section through line X-X of FIG. 41;

FIG. 43 to 45 are perspective views showing three steps in the operation of the device according to FIGS. 41 and 42.

### DETAILED DESCRIPTION OF THE DRAWINGS

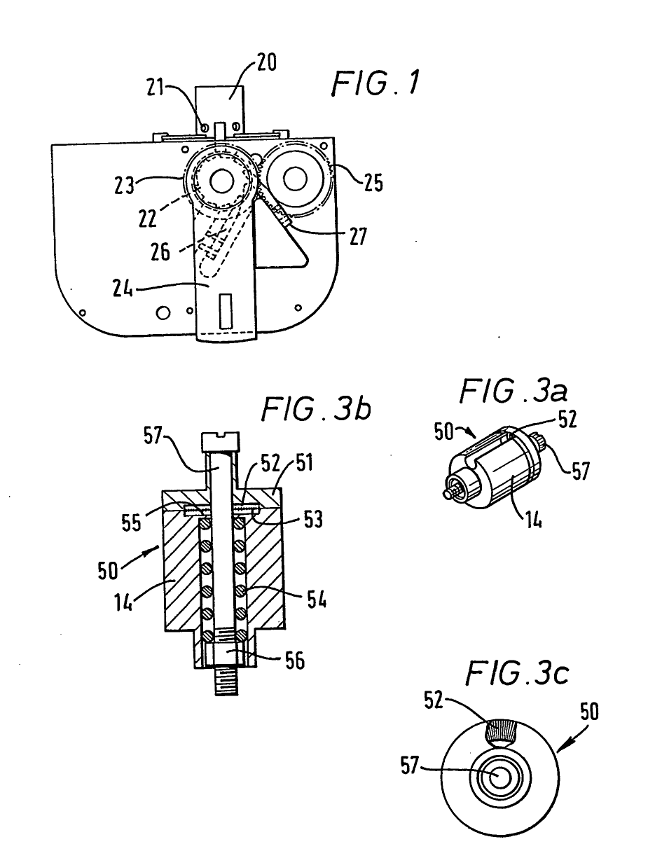

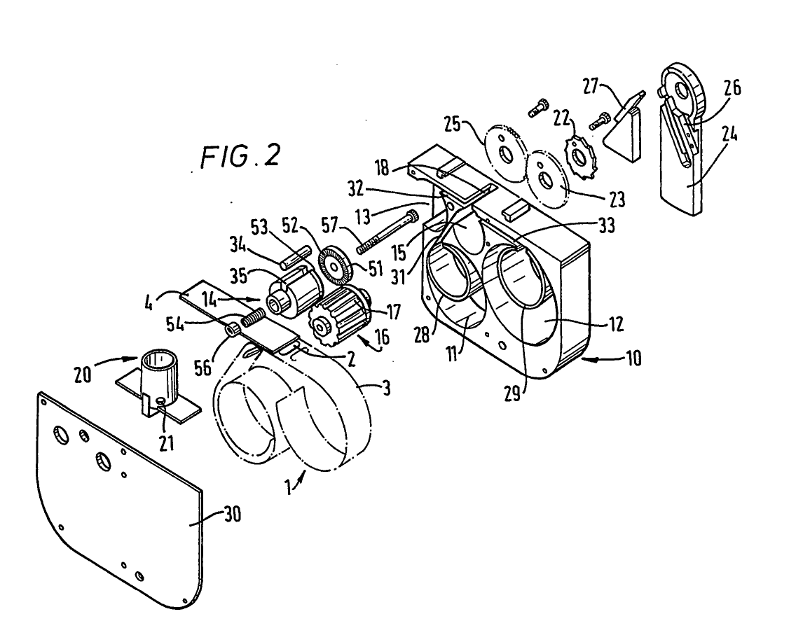

Referring now to FIGS. 1, 2 and 3a to 3c, these show an inhalation device in which is mounted a flexible strip 1 defining a plurality of pockets 2 each of which contains a dose of medicament e.g. having a formulation as described hereinbefore in any of the Examples 1-12 which can be inhaled, in the form of a powder. The strip 1 comprises a base sheet 3 in which blisters are formed to define the pockets 2, and a lid sheet 4 which is hermetically sealed to the base sheet 3 except in the region of the blisters, in such a manner that the lid sheet and the base sheet can be peeled apart. The sheets are sealed to one another over their whole width except for leading end portions thereof where they are preferably not sealed to one another at all. The lid and base sheets are each preferably formed of a plastics/aluminium laminate, and the lid and base sheets are preferably adhered to one another by heat sealing. By way of example, the lid material may be a laminate consisting of 50 gsm bleach kraftpaper/12 micron polyester (PETP) film/20 micron soft temper aluminium foil/9 gsm vinylic peelable heat seal lacquer (sealable to PVC), and the base material may be a laminate consisting of 100 micron PVC/45 micron soft temper aluminium foil/25 micron orientated polyamide. The lacquer of the lid material is sealed to the PVC layer of the base material to provide the peelable seal between the lid and base sheets.

The strip 1 is shown as having elongate pockets which run transversely with respect to the length of the strip. This is convenient in that it enables a large number of pockets to be provided in a given strip length. The strip may, for example, be provided with sixty or one hundred pockets, but it will be understood that the strip may have anysuitable number of pockets.

The inhalation device comprises a body 10 defining three storage chambers, namely a chamber 11 in which the strip 1 is initially housed and from which it is dispensed, a chamber 12 for receiving the used portion of the base sheet 3, and a chamber 13 within which the used portion of the lid sheet can be wound up on a wheel 14. The chambers 11 and 12 contain respective curved leaf springs 28 and 29, the purpose of which is described below. The body defines a further chamber 15 which houses an index wheel 16. This has a plurality of grooves 17 extending parallel to the axis of the wheel 16. The grooves are spaced at a pitch which is equal to the distance between the centre lines of adjacent pockets 2. The chambers 11, 12, 13 and 15 are closed by a lid 30. The chamber 15 communicates with the chambers 11, 12 and 13 via passages 31, 33 and 32 respectively.

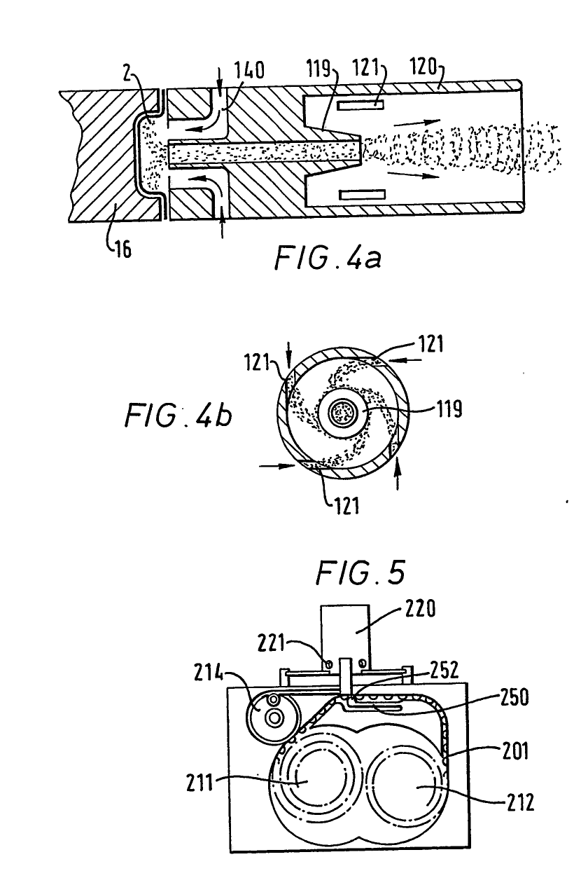

The chamber 15 communicates via a slit 18 which, in turn, extends upwardly within a mouthpiece 20. The slot 18 also communicates with air inlets, as will be described below with reference to the specific mouthpiece shown in FIGS. 4a and 4b. The mouthpiece 20 is provided with additional air inlets 21 shown here in the form of a pair of circular apertures, though they may be of some other shape, as they are in FIGS. 4a and 4b. The primary purpose of the additional air inlets 21 is to provide additional air to the user and thus reduce the resistance to inhalation, though they may serve one or more additional purposes, as they do in FIG. 4a and 4b and as is described below with reference to those Figures.

A means is provided by which the user can rotate the index wheel and the lid wheel in steps of a predetermined size. This means comprises a ratchet wheel 22 and a gear wheel 23, both connected to rotate in unison with the index wheel 16, a lever 24 arranged to rotate about the same axis as the ratchet wheel 22 and gear wheel 23, but independently thereof, and a gear wheel 25 which meshes with the gear wheel 23 and is arranged to rotate the lid wheel 14. The lever 24 carries a pusher arm 26, the end of which is arranged to engage the teeth of the ratchet wheel 22. The teeth of the ratchet wheel are also engaged by a pawl 27 fixedly secured to the body 10. For reasons which will become apparent from the description below of the operation of this embodiment, the gear wheel 25 is not connected directly to the lid wheel 14, but is connected via a slipping clutch 50 which is housed within the lid wheel 14. The effect of the provision of this clutch is that slipping occurs between the lid wheel and the gear wheel 25 when the force required to rotate the lid wheel exceeds a predetermined amount.

The clutch 50 comprises a disc 51 provided with radially extending serrations 52, or other surface roughness, which is held in engagement with a similarly serrated or roughened surface 53 provided on an end face of the lid wheel 14 by a compression spring 54. The spring 54 bears at one end against an inwardly directed surface 55 of the lid wheel and at the other end against a nut 56 threaded on a bolt 57.

The device described above can be made so as to be reusable after thedoses of medicament contained in the pockets 2 have all been dispensed. In that case, provision can be made for the user to gain access to the interior of the device, for example by removing the lid 30, so as to insert therein a fresh strip 1, for example in a cassette. Alternatively, however, the device may be made to be disposable once the strip 1 with which it is supplied has been used up.

In either event, when the device is first used the bulk of the strip 1 is within the chamber 11, kept in a relatively tight reel by the leaf spring 28, with a short portion at the leading end thereof passing out of the chamber 11 through the passage 31 to the index wheel 16. The foremost part of the leading end of the strip is peeled apart so that the leading end of the lid sheet 4 can be secured to the lid wheel 14, and so that the leading end of the base sheet 3 can enter the passage 33. The end of the lid sheet 4 is held in place on the lid wheel 14 by means of a key 34 which is a force fit in a slot 35 in the wheel 14. A user desiring to use the device pushes the lever 24 in an anticlockwise direction, as viewed in FIG. 1, so that the pusher arm 26 urges the ratchet wheel 22 through an angle equal to the angular distance between two adjacent teeth. This causes the ratchet wheel 16 to rotate by an angular amount equal to the pitch of the groove 17 thereof and thus equal to the distance between two adjacent pockets 2 in the strip 1

This brings a pocket 2 opposite the slot 18 in the body 10. Since the ratchet wheel 22 and gear wheel 23 move in unison, and since the gear wheel 25 meshes with the gear wheel 23, movement of the lever 24 also causes the lid wheel 14 to rotate. This peels a sufficient portion of the lid sheet 4 away from the base sheet 3 to expose the contents of the pocket 2 which is being brought into alignment with the slot 18.

When the user inhales through the mouthpiece 20 the flow of air which this produces entrains powder from the opened pocket, so that the powder is inhaled by the user. One way in which this can occur is explained in more detail below with reference to the embodiment of mouthpiece shown in FIGS. 4a and 4b. Each time the above procedure is repeated a further length of lid sheet is wrapped around the lid wheel 14 and a further length of base sheet enters chamber 12 through passage 33. The leaf spring 29 therein ensures that the base sheet is coiled up and does not snag on the wall of the chamber 12.

One effect of winding up the lid sheet on the lid wheel 14 is that the external diameter of the wheel plus the sheet wound thereon gradually increases. Were it not for the use of a slipping clutch to connect the gear wheel 25 to the lid wheel 14 this would have the result that successive operations of the lever 24 would try to cause a progressively longer length of lid sheet to be wound on to the lid wheel. The slipping clutch 50, however, avoids this effect, the clutch slipping each time by an amount sufficient to ensure that for every operation of the lever the amount of lid sheet wound on is precisely equal to the pitch of the pockets 2.

FIGS. 4a and 4b show a portion of the index wheel 16 with a pocket 2 therein, in conjunction with a mouthpiece which differs slightly from the mouthpiece 20 shown in FIGS. 1 to 3, and which is denoted by reference numeral 120. The mouthpiece 120 has air inlets 140, to which reference in general terms has already been made in connection with FIGS. 1 to 3, and a central powder outlet 119, one end of which is open to the pocket 2 and the other end of which opens into the interior of the mouthpiece 120.

When a user inhales through the mouthpiece 120 this causes air to flow in through the inlets 140 and thence through the pocket 2, into the powder outlet 119, and out through the mouthpiece 120. By thus directing the flow of air through the pocket 2, efficient entrainment of powder in the airflow is achieved, with consequent efficient emptying of the pocket. The mouthpiece 120 is provided with additional air inlets 121, shown here by way of example as being four in number, which open tangentially into the mouthpiece. When the user inhales air is drawn into the mouthpiece not only through the air inlets 140 but also through the air inlets 121, and the air entering through the inlets 121 produces a swirling airflow which helps to distribute powder effectively within the airflow and reduce the extent to which powder is deposited on the inside of the mouthpiece. This also helps to break up any aggregates of powder which may be present in the blister.

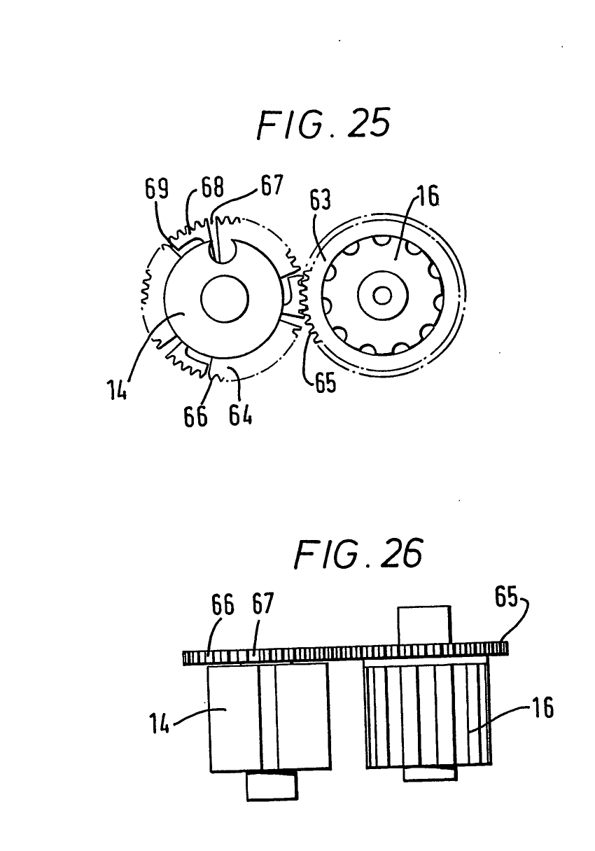

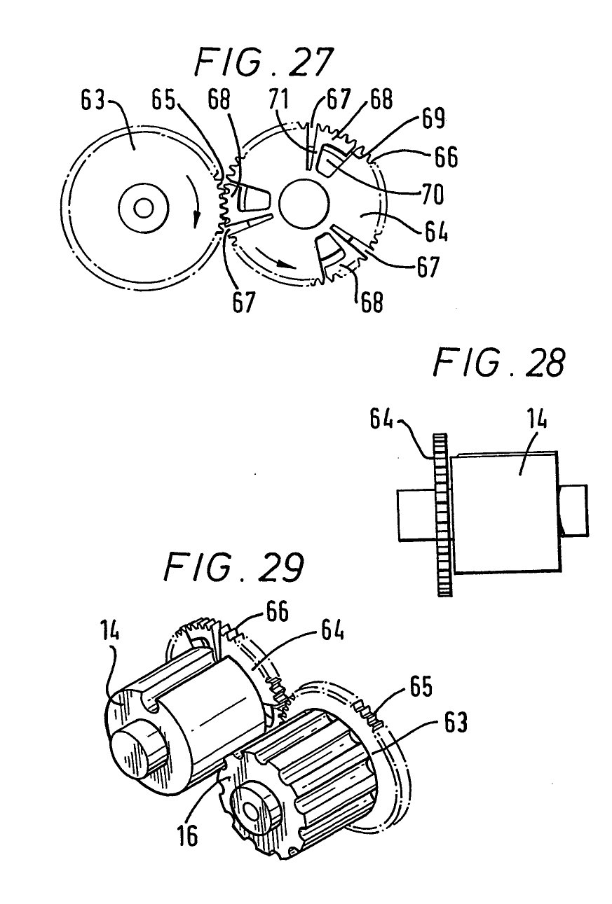

An alternative clutch arrangement is shown in FIGS. 25 to 29. In this, the index wheel 16 and the lid wheel 14 have respective toothed gear wheels 63 and 64 secured to them for rotation therewith. The direction of rotation is indicated by arrows in FIG. 27.

Gear wheel 63 has a toothed surface 65, with the teeth being provided continuously all the way round the surface 65 and at a constant pitch. By contrast, the gear wheel 64 has a toothed surface 66 from which some teeth are missing by virtue of the provision of radially extending slots 67. The circumferential width of each slot at the surface 66 is equal to one tooth pitch. The drawings show three such slots, but it should be understood that there could instead be one slot, two slots, or more than three slots. To one side of each of the slots 67, in fact upstream of each slot as considered in the direction of rotation of the gear wheel 64, a toothed section 68 is defined between the slot 67, and a narrow slit 69. The radially inner end of each slit 69 communicates with an aperture 70, so that each toothed portion 68 is connected to the remainder of the gear wheel 64 only by an arm 71. The gear wheel 64, or at least those portions thereof which provide the arms 71, is made of a material which permits the toothed portions 68 to flex resiliently back and forth in a circumferential direction. The rest position of the portions 68 is as shown in the drawings, but when a force is applied to a portion 68 in the direction of rotation of the gear wheel 64, the portion 68 can move so as to close the gap 67 at the radially outer end. This has the effect that a tooth is then “missing” not at the end of the slot 67 but at the end of the slit 69.

When the circumferential force applied by the gear wheel 63 to the gear wheel 64 is below a predetermined level the toothed portions 68 remain in their rest positions and the gear wheel 64 behaves just as if it had a continuous toothed surface like that of gear wheel 63. However, if the load exceeds a predetermined value, each time a toothed section 68 meshes with the gear wheel 63 it is moved circumferentially to close up the slot 67 at its outer end and open the slit 69. This movement of the toothed section 68 by a distance equal to the tooth pitch has the effect of producing slippage of the gear wheel 64 with respect to the gear wheel 69 equal to one tooth pitch. In this way, the illustrated arrangement is able to permit a total slippage of the gear wheels with respect to one another by a maximum of a distance equal to three times the tooth pitch per revolution, and hence a corresponding slippage of the lid wheel and index wheel with respect to one another. As will be appreciated, providing more or fewer toothed sections than the three illustrated will permit more or less than this maximum slippage.

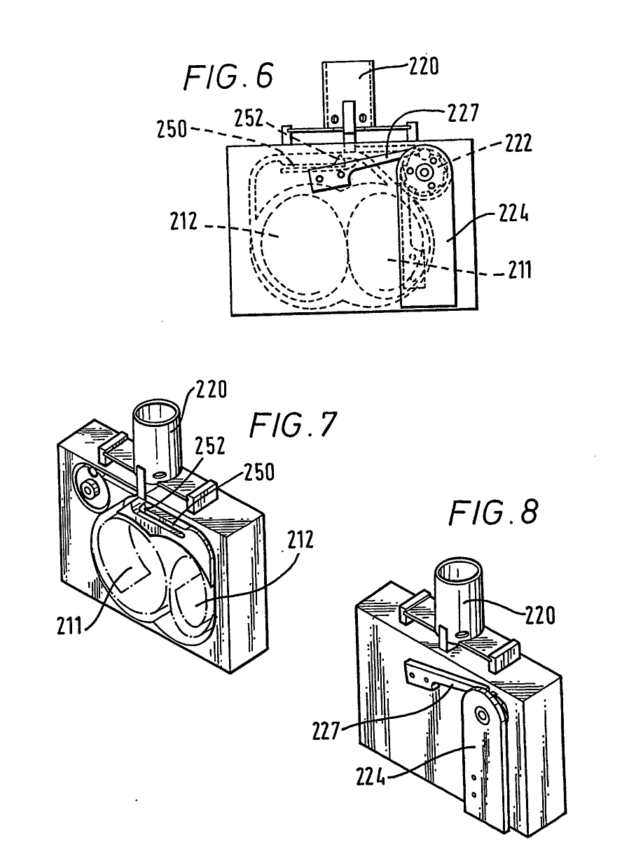

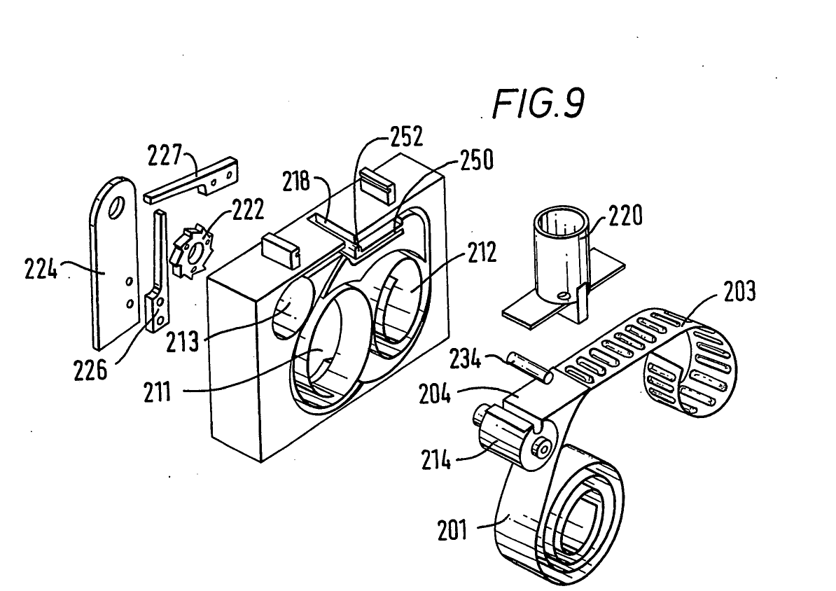

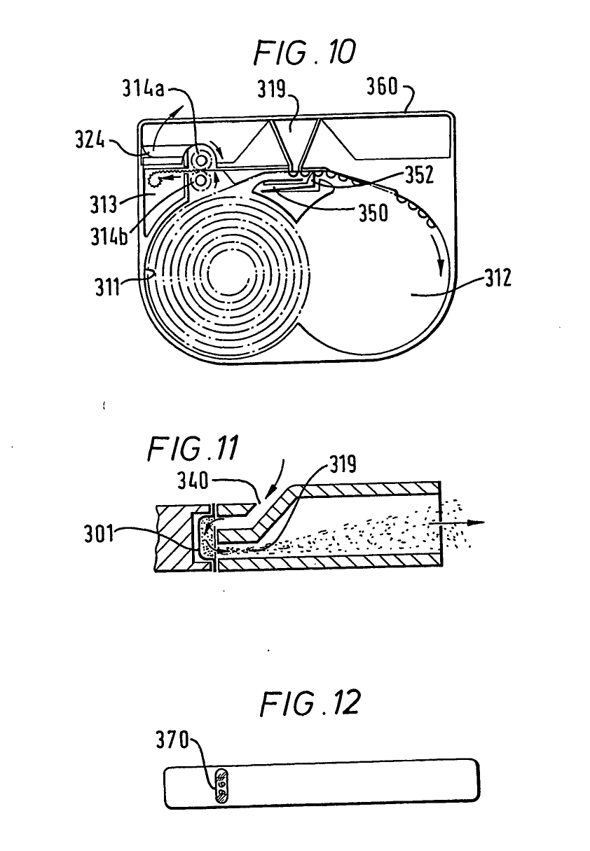

A second embodiment of the inhalation device according to the invention is shown in FIGS. 5 to 9. This is intended for use with a strip 201, similar to the strip 1 used in the first embodiment except as regards the spacing of the pockets (for which see below). In many respects the second embodiment resembles the first embodiment, and components in the second embodiment which correspond in general terms to particular components in the first embodiment are denoted by the same reference numerals, but with the addition of 200. The main difference between the first embodiment and the second embodiment is that in the latter there is no index wheel corresponding to the index wheel 16 of the first embodiment. Instead, indexing of the strip 1, to ensure that each operation of the lever advances the strip by an amount equal to the pitch of the pockets, is achieved by a resiliently flexible arm 250 terminating in a tooth 252 which engages between adjacent pockets. Each time the lever 224 is operated the arm 250 is resiliently depressed as a pocket slides past the tooth 252 thereof, and the tooth then springs back into engagement with the strip to the rear of the pocket which has just passed it.

It will be appreciated that, as in the case of the first embodiment, the diameter of the lid wheel 214 with the lid sheet thereon gradually increases during operation. Since a slipping clutch cannot be used in this embodiment the effect just described is compensated by having the spacing of the pockets 2 gradually increasing towards the rear end of the strip.

One other difference which will be noted between the first and second embodiments, is that in the latter the chambers 211 and 212 form a single composite chamber, unlike the separate chambers 11 and 12 in the first embodiment. However, this need not be so, and the first embodiment could use a single composite chamber and the second embodiment could use separate chambers.

FIGS. 10 to 12 show a third embodiment. In many respects this resembles the second embodiment, and components in the third embodiment which correspond in general terms to components in the second embodiment are denoted by the same reference numerals but with the addition of a further 100.

One difference which will be observed between the second and third embodiments is that in place of the lid wheel 114 a pair of wheels 314a and 314b are employed, with the lid sheet being gripped in the nip between the wheels 314a and 314b, which act as a mangle. These wheels are knurled or otherwise roughened to improve the grip between the wheels and the lid sheet. The used lid sheet is not wound up but is fed into a chamber 313, so that no problem arises, as it does in the first two embodiments, with the lid wheel attempting to wind up progressively longer lengths of lid as operation of the device continues.

FIG. 11 shows the mouthpiece to be of a somewhat different design to that shown in FIGS. 4a and 4b. The mouthpiece is shown as having a single air inlet 340 in place of the pair of air inlets 140, and the powder outlet 119 of FIGS. 4a and 4b is replaced by a mouthpiece portion 319 of reduced width. It should be understood, however, that the device shown in FIGS. 10 to 12 could be modified so as to incorporate a mouthpiece more closely resembling FIGS. 4a and 4b.

FIG. 10 shows the device as being provided with a hinged cover 360, and such cover could be provided for either of the first two embodiments. FIG. 12 shows the device as having a window 370 through which indicia on the strip can be viewed. By printing the strip with numbers or other indicia which correlate with the number of pockets from which powder has been dispensed, or alternatively is to be dispensed, the user is provided with an indication of how many doses have been used or, alternatively, how many doses remain. Another possibility is to use a dose counting device driven by one of the rotating components of the inhalation device. It should be noted that similar indicia and means for viewing those indicia could be provided in all the embodiments. FIGS. 13 to 16 show a further embodiment of the invention. This is similar in the principle of its operation to the first embodiment, and components in the fourth embodiment which correspond in general terms to components in the first embodiment are denoted by the same reference numerals but with the addition of 400.

As in the first embodiment, the device receives a flexible strip, here denoted as 401, comprising a base sheet 403 in which pockets 402 are defined and a lid sheet 404. The strip 401, is shown most clearly in FIG. 35. The lid sheet 404 has a loop 404a formed at the leading end thereof for engagement over a post 471a extending upwardly from a toothed wheel 471 (described below). The base sheet has a lead portion 403a of reduced width for engagement in a slot 470a formed in the base winding wheel 470 (described below). The leading end portions of the base sheet and lid sheet are not sealed together, as can be seen in FIG. 35.

The body 410 comprises a base 410a and a top 410b both of generally circular shape. When the device is assembled the base and top are snap-fitted together. The body defines a single internal chamber within which the strip 401 is housed and within which are also housed a wheel 414 for winding up the used portion of the lid sheet 404, a base winding wheel 470 and an index wheel 416. The index wheel 416 is hollow and an index ratchet wheel 422 is housed within it. All the wheels just mentioned are mounted in the chamber defined by the body, for rotational movement with respect thereto. A pawl 470b is attached to the body 410 and engages the teeth of the base winding wheel 470 to prevent the wheel moving anticlockwise, thus ensuring that the strip 401 can only proceed forwards through the device.

The lid winding wheel 414 is formed in two parts, namely a toothed wheel 471 having teeth 472 and a shaft 473, and a collapsible wheel 474 having a hollow central shaft 475 and a plurality of resilient arms 476, for example, as shown, eight such arms, extending from the central shaft 475 each at an angle to a radius. The toothed wheel 471 has a lug 477 which engages in a corresponding notch in the shaft 475 so that the wheels 471 and 474 rotate in unison.

The hollow index wheel 416 has external teeth 478 which mesh with the teeth of the base winding wheel 470 and the teeth of the wheel 471. Ratchet teeth 479 are formed on the internal walls of the index wheel 416, and the index ratchet wheel 422 has two pawls 480 which engage the ratchet teeth 479.

The device further comprises a lever 424 which comprises an arcuate wall 481 with a finger tab 482, and an arm 483 which extends inwardly from the wall 481 and carries an arcuate array of teeth 484 at its distal end. The lever is pivotally mounted to the centre of the base 410a for movement about an axis which is at the centre of the pitch circle of the teeth 484, the teeth 484 mesh with the teeth 485 on the index ratchet wheel 422.

A manifold 486 provides communication between the chamber within the body 410 and a mouthpiece 420. The manifold has a powder outlet 419 and also has a passageway 487 to allow used lid strip 404 to pass to the collapsible wheel 474. Optionally, a roller 488 may be provided to guide the strip 404 into the passageway 487.

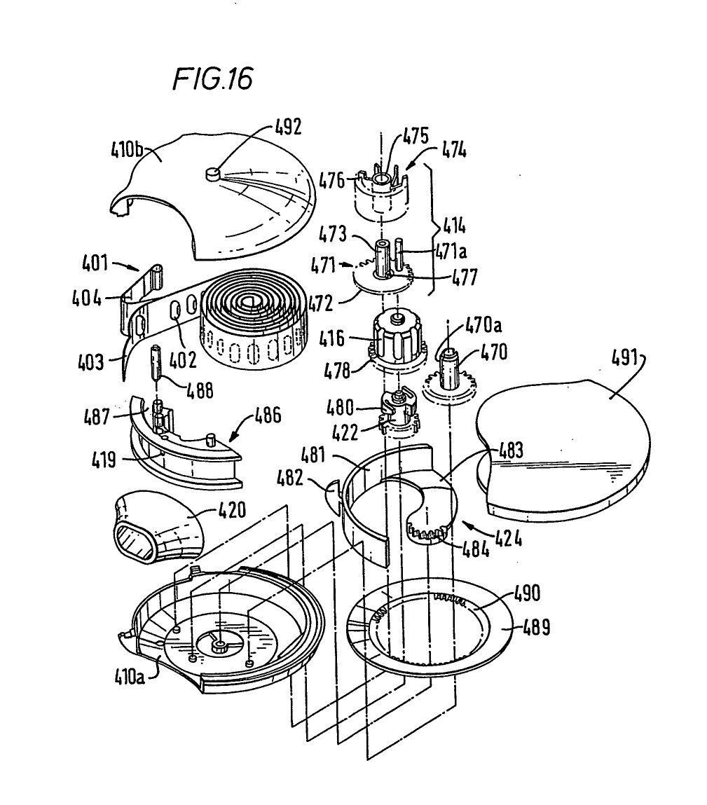

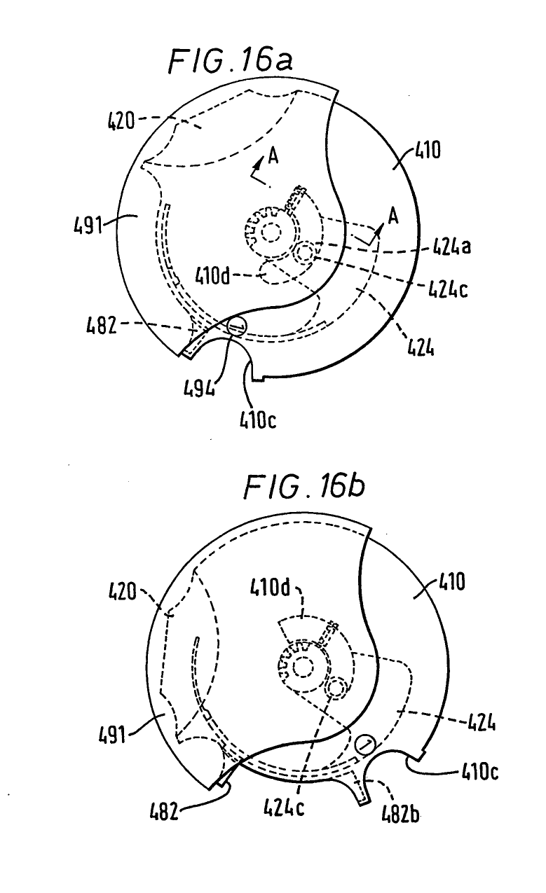

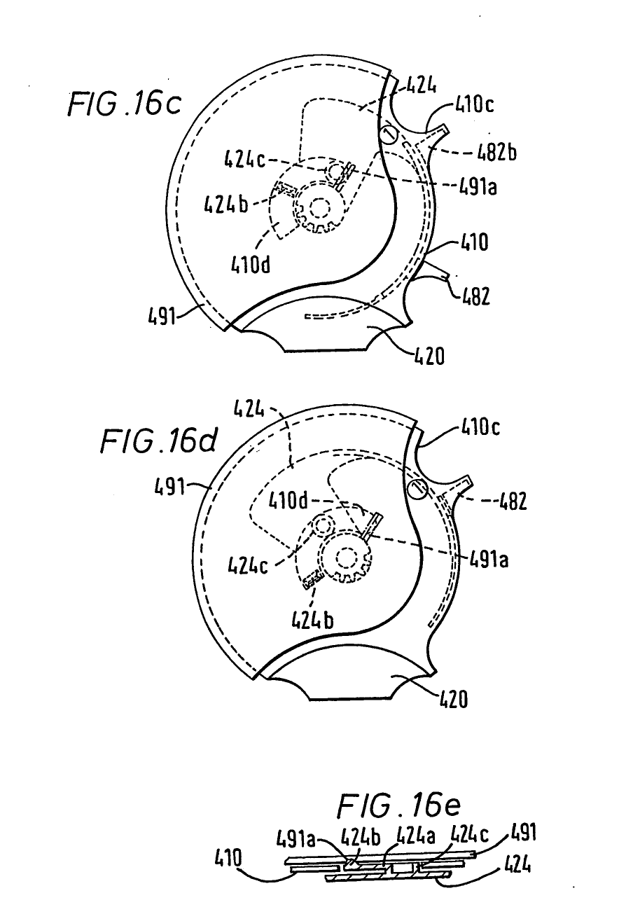

A dose monitor ring 489 having teeth 490 is arranged to be rotatable within the body base 410a. On its lower surface this bears indicia (not visible in the drawings) which can be viewed by the user through a window 494 in the body 410. It will be noted from FIGS. 16a to 16d that the window can be seen both when the cover 491 (see below) is closed and when it is open. The indicia indicate either exactly or approximately the number of doses left (or the number of doses used, if preferred). The ring 489 is rotated by virtue of the fact that its teeth 490 are engaged by the teeth 478 of the index wheel.

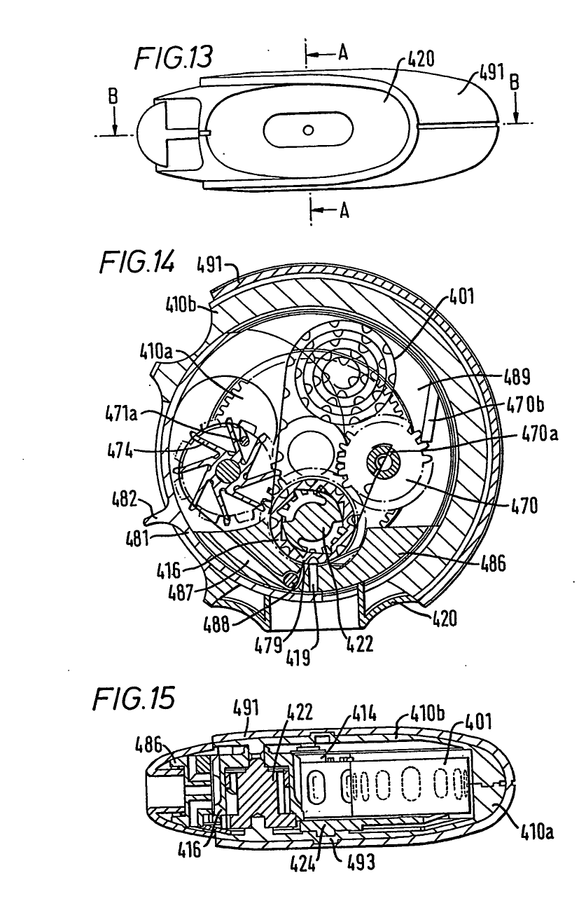

The device is provided under a cover 491 which is pivotally mounted on the body 410 by means of a lug 492 on the body top 410b and a corresponding lug 493 on the body base 410a. The cover is pivotal between an open position (shown in FIG. 14) in which the mouthpiece is exposed and a closed position in which it is not, as is described more fully below.

In operation, the user moves the cover 491 to its open position and then presses on the finger tab 482 of the lever 424 to cause it to move as the lever pivots. This makes the index ratchet wheel 422 rotate which, via the pawls 480, causes the index wheel 416 also to rotate. Rotation of the index wheel 416 produces rotation of both the base winding wheel 470 and the lid winding wheel 414, thus peeling the base sheet and lid sheet apart over a distance sufficient to expose a previously unopened pocket 402 opposite the end of the powder outlet 419 in the manifold. The patient can then inhale through the mouthpiece, as in the preceding embodiments.

Successive stages in the operation of the device are shown in FIGS. 16a to 16d. The device is in its closed position in FIG. 16a. The finger tab 482 of the lever 424 is at this stage in a recess 482b formed in the body 410 (seen more clearly in FIGS. 16b and 16c). The cover 419 is held stationary as the body 410 is rotated anticlockwise, a recess 410c being provided in the periphery of the body to enable the user to insert a finger for this purpose. The device is thus moved to the partly open position shown in FIG. 16b. During this process the lever 424 remains stationary with respect to the cover 491. This is achieved by the lever being provided internally with a resilient arm 424a the tip 424b of which engages in a recess 491a in the cover 491. The arm 424a is attached to the lever 424 via a cylindrical member 424c. As viewed in FIG. 16a, the arm 424a extends anticlockwise from the member 424c over an arc of about 90.degree. The cylindrical member 424c is guided in an arcuate slot 410d formed in the body 410. The slot 410d extends through an arc of about 180.degree., and in FIG. 16a the member 424c is shown as being approximately half way along its length. In FIG. 16b it is shown as being at one end.

The user continues to rotate the body 410 from the position shown in FIG. 16b to the position shown in FIG. 16c. During this further rotation tip 424b of the arm 424a jumps out of the recess 491a. This occurs because, with the member 424c at one end of the slot 410d, movement of the body 410 carries the member 424c with it in an anticlockwise direction and hence compels the arm 424a likewise to move anticlockwise. The user then moves the lever 424 by pushing on the finger tab 482 to cause it to rotate anticlockwise through the position shown in FIG. 16c to the position shown in FIG. 16d where the finger tab 482 re-enters the recess 482b. The steps thus far described both expose the mouthpiece 420 and open a fresh blister. The device is therefore now ready for the user to inhale.

After use, the body 410 is rotated clockwise, the lever 424 moving in unison with the body, to bring the device back to the position of FIG. 16a.

It will be noted that the collapsible wheel 474 in effect assumes the function of the clutch in the first embodiment. As more lid sheet is wound onto the wheel 474 the arms 476 gradually flex inwardly, and the effect is to keep the external diameter of the reel of wound up lid sheet substantially constant, while the internal diameter thereof gradually decreases.

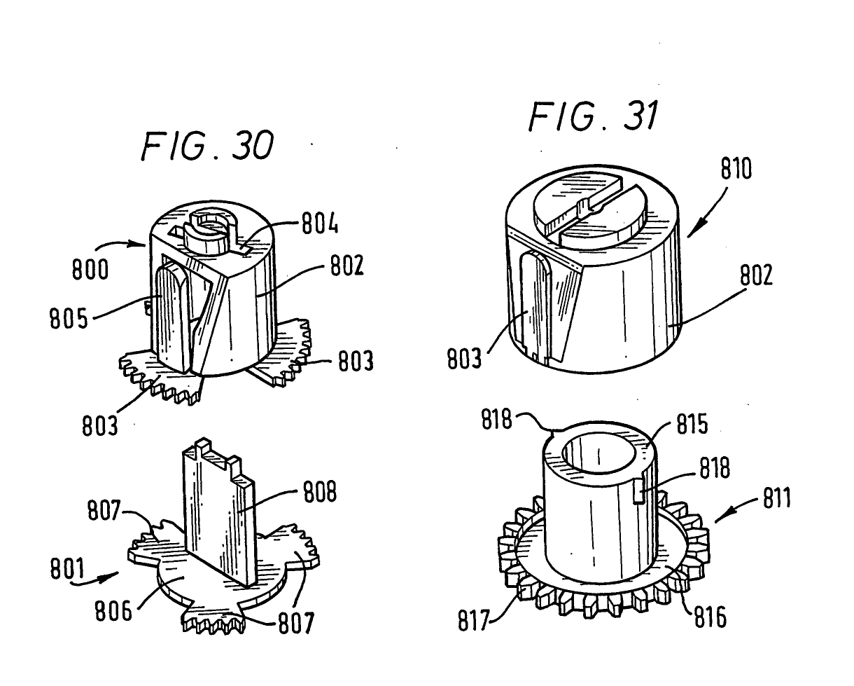

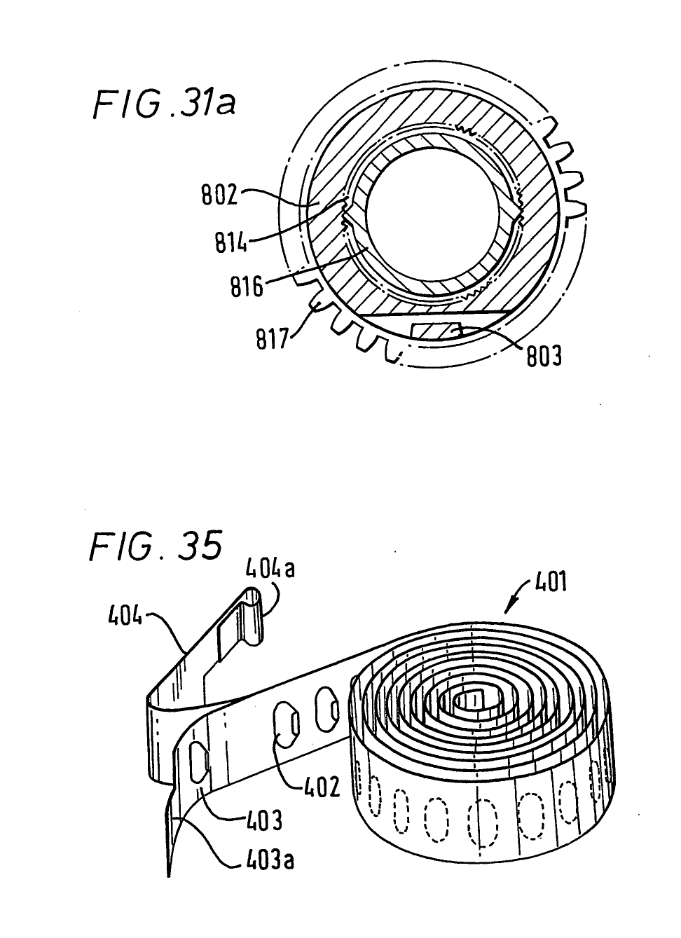

Instead of the wheel 414 with its collapsible wheel 474 it is possible to use the alternative structure shown in FIG. 30 or that shown in FIGS. 31 and 31a. The principle of operation of the structure shown in FIG. 30 is very similar to that of the clutch arrangement shown in FIGS. 25 to 29. The structure of FIG. 30 comprises two components 800 and 801. The component 800 comprises a generally cylindrical hollow housing 802 open at its lower end and three arcuate arrays of teeth 803. The cylinder 802 has a slot 804 extending through the upper surface thereof, and a post 805 for receiving the leading end of the lid sheet. The component 801 comprises a disc 806 provided with three arcuate arrays of teeth 807, and an upright member 808 extending upwardly from the disc 806. The member 808 is formed of a material, example a plastics material, which is resilient in torsion.

The two components 800 and 801 are snap-fitted together so that the upper end of the member 808 is received in the slot 804 and cannot rotate with respect thereto. The arrays of teeth 803 and 807 are coplanar and alternate with one another. The teeth 803 and 807 mesh with the teeth 478 of the index wheel. Each array 807 is separated from one of the adjacent arrays 803 (but not from the other) by a gap equal to one tooth. Thus, there are three gaps, each of one tooth width, around the assembled arrays. Because the member 808 can flex in torsion, the disc 806 is free to move back and forth between a position in which the gaps are each on one side of a respective array 807 and a position in which the gaps are each on the other side of a respective array 807. This has the effect of producing slippage of the structure shown in FIG. 30 with respect to the index wheel.

The structure shown in FIG. 31 is a slipping clutch. It comprises two components 810 and 811, snap-fitted together. The component 810 comprises a generally cylindrical housing 812 open at its lower end and having a post 813 for receiving the leading end of the lid sheet. The interior of the housing 812 is provided with longitudinally extending serrations 814, as can be seen in FIG. 31a. The component 811 comprises a cylinder 815 which extends upwardly from a disc 816 provided with teeth 817. The teeth 817 mesh with the teeth 478 of the index wheel. The cylinder 815 is provided on its outer surface with a pair of pips 818 which are in interfering engagement with the serrations 814. When the rotational force applied by the component 811 to the component 810 is below a predetermined level the components rotate together. However, the cylinder is made of a material, for example a plastics material, which can deform radially, and when the rotational force exceeds the predetermined level such deformation takes place, permitting the pips 818 to move over the serrations 814.

Although in the embodiment of FIGS. 13 to 16, with or without the modifications of FIGS. 30 and 31, the base sheet is wound up as well as the lid sheet, it is not necessary for there also to be a slipping clutch or the like between the index wheel and the base winding wheel. The diameter of the base winding wheel is so chosen that initially the base sheet is wound up only very loosely, and the tightness with which the sheet is wound increases during operation but without ever reaching an unacceptable level. In theory, the base sheet could be wound up precisely via a slipping clutch or the like, with the lid sheet being only loosely wound, but in practice it is much easier to wind up the lid precisely because it is flat and because it is thinner than the base sheet.

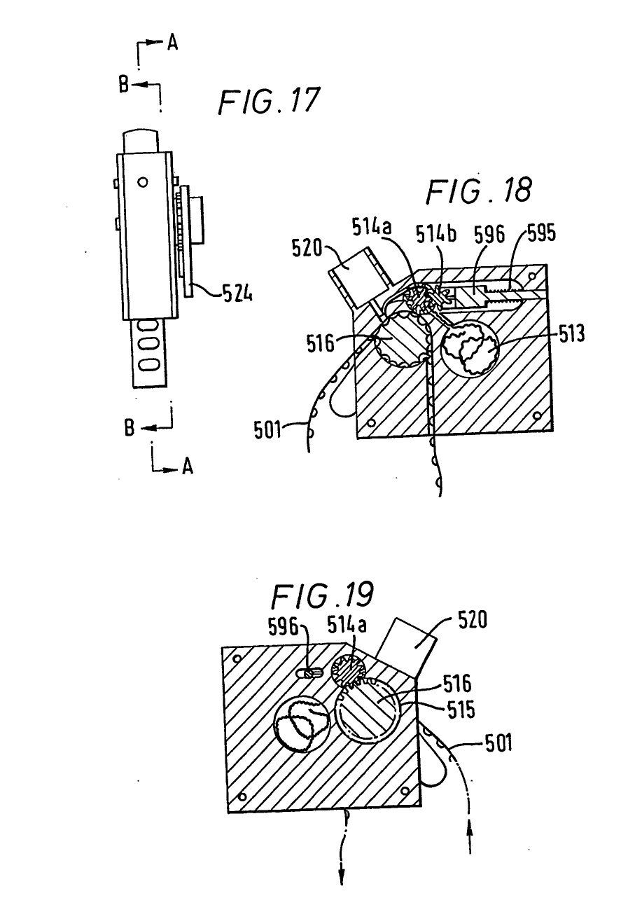

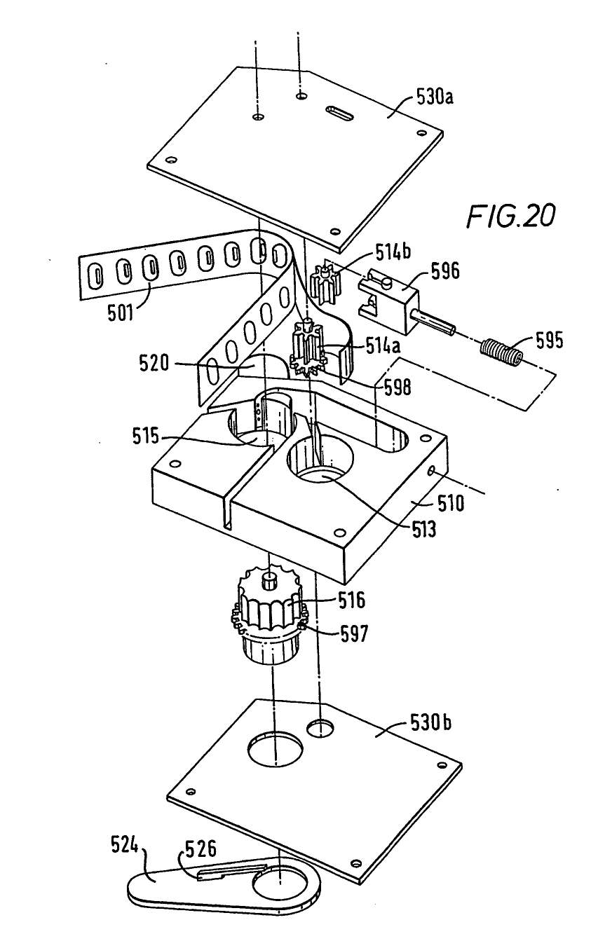

FIGS. 17 to 20 show in diagrammatic form the main operative parts of a device which has some similarities to those shown in FIGS. 10 to 12, i.e. it is a mangle device. However, it is to be understood that FIGS. 17 to 20 do not show a complete device, the chamber for the unused strip and the used base material being omitted. Components in this embodiment which correspond in general terms to particular components in the embodiment of FIGS. 10 to 12 are denoted by the same reference numerals, but with the addition of a further 200.

The device of FIGS. 17 to 20 comprises a pair of wheels 514a and 514b which have meshing teeth formed thereon and which act as a mangle engaging the used lid material. This material is fed into a chamber 513. The wheel 514b is an idler wheel and is urged into engagement with the wheel 514a by a compression spring 595 which acts on a carrier 596 which carries the wheel 514b. The wheel 514a has a ring of gear teeth 598 which mesh with teeth 597 formed on an index wheel 516 which performs the same indexing function as the index wheel 16 in the first embodiment and is rotatable in a chamber 515. The chambers are formed in a body 510 and lids 530a and 530b are secured to opposite sides of the chamber. Inhalation is through a mouthpiece 520. The device is operated by a lever 524 which turns the index wheel 516 via a pusher arm 526.

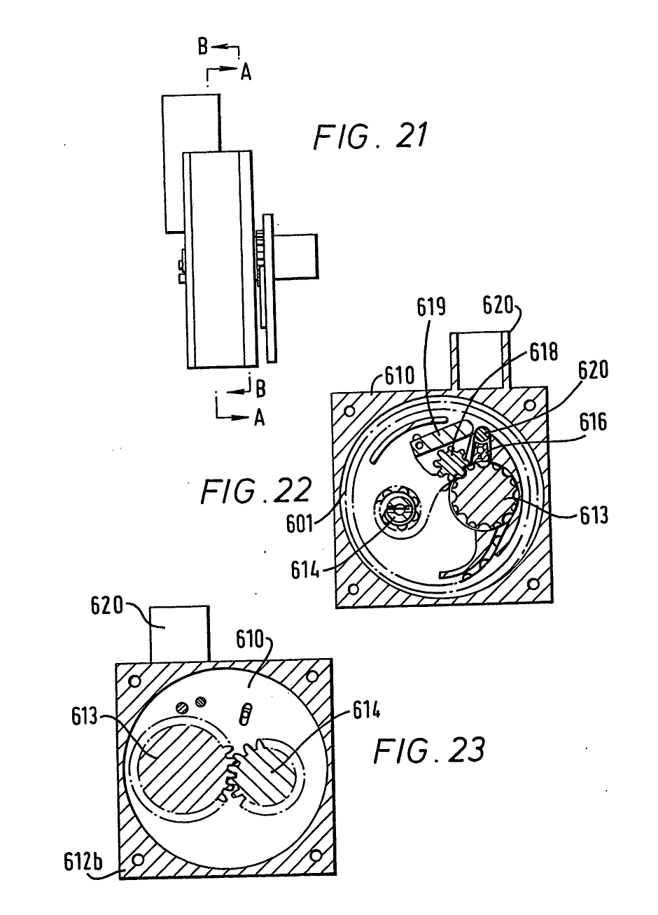

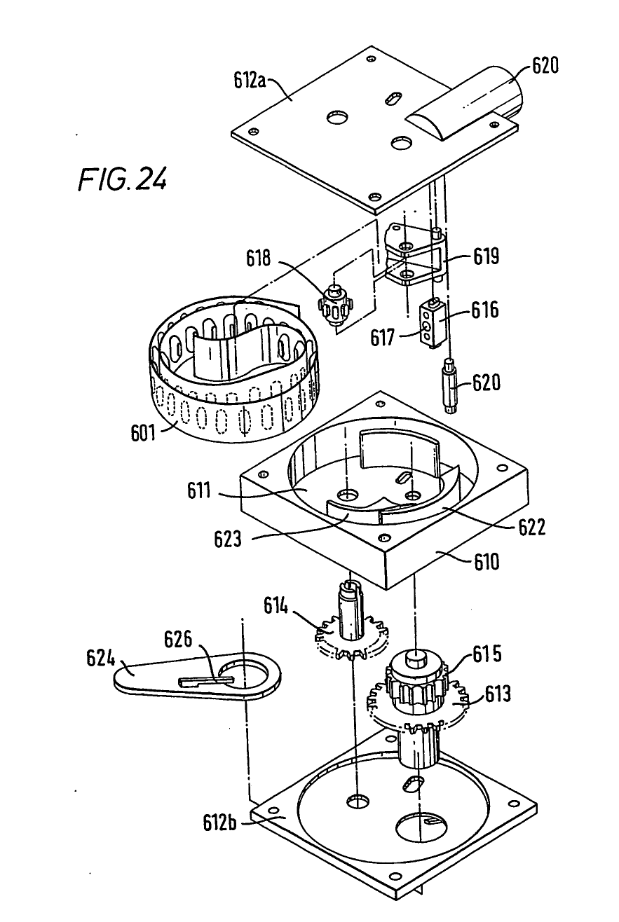

The embodiment shown in FIGS. 21 to 24 is another type of mangle device, but one in which both the lid and base sheets pass through the wheels of the mangle.

The embodiment of FIGS. 21 to 24 comprises a body 610 defining a substantially circular chamber 611 and having lids 612a and 612b secured thereto. Within the chamber 611 an index wheel 613 and a base and lid winding wheel 614 are rotatably mounted, the wheels 613 and 614 having gear teeth which mesh with one another. The index wheel 613 has grooves 615, and a lid gripper wheel 618, rotatably carried in a carrier 619 is also mounted adjacent the grooves 615, downstream of the manifold 616. A roller 620 is mounted behind the manifold 616 to guide the lid sheet. Flexible strip 601 is provided in the chamber 611, the main part of the strip being initially coiled up around the internal wall of the chamber. The leading end of the strip passes between guide members 622 and 623 over part of the circumference of the index wheel 613, with the powder containing pockets thereof engaged in the grooves 615. At the point where the strip meets the manifold 616 it is peeled apart, and the lid sheet passes behind the manifold and over the roller 620 while the base sheet passes between the index wheel and the manifold. After the manifold both sheets pass between the index wheel and the lid gripper wheel 618, and are gripped thereby. The front end of the strip is fixed in the base and lid winding wheel 614.

In use, the strip 601 is advanced by rotating the index wheel, by means of a lever 624, via a pusher arm 626, which causes corresponding rotation of the base and lid winding wheel. This winds up the base and lid, initially loosely, though increasing in tightness as the operation proceeds, but without, however, the tightness ever reaching an unacceptable level. The lid and base sheets are peeled apart where the strip meets the manifold 616, presenting a fresh pocket of powder to the powder outlet 617. Inhalation is via a mouthpiece 620.

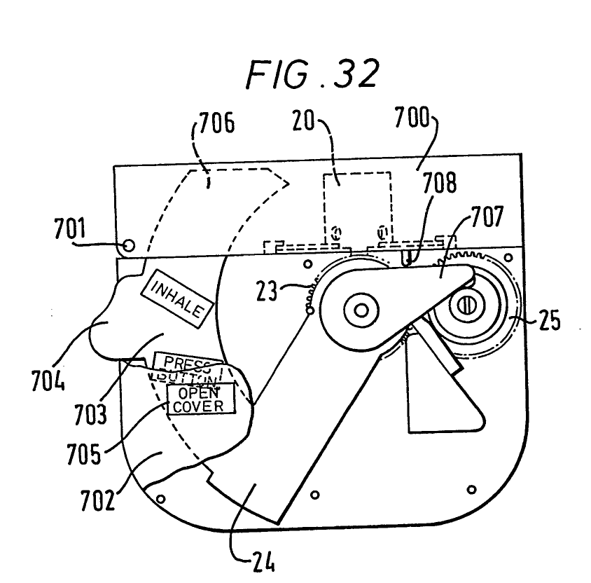

FIGS. 32 to 34 show an embodiment of the invention incorporating, as a further feature, indicia which instruct the user as to the successive steps which the user is to take to operate the device. Apart from the indicia, the device is largely the same as the embodiment shown in FIGS. 1 to 3, and the same reference numerals are used for the corresponding components. However, there are some additional components, as will be apparent from the following description.

The device shown in FIGS. 32 to 34 has a cover 700 which is pivotally connected to the remainder of the device for pivotal movement about an axis 701. The gear wheels 23 and 25 and the associated components are covered by a rear wall 702. This extends over the whole of the rear of the device, but in the drawings all except a small portion thereof is shown broken away for ease of understanding. The lever 24 is provided with an arcuate extension 703, on an edge whereof is formed a cam 704. The extension 703 carries indicia in the form of instructions to the user, in this case the legends “OPEN COVER”, “PRESS BUTTON”, “INHALE”. When the lever 24, and hence the extension 703, are in particular positions a respective one of these legends is visible through a window 705 in the rear wall 702. The distal end of the extension 703 constitutes a button 706. The end of the lever 24 remote from the extension 703 carries a tongue 707 pivotal therewith.

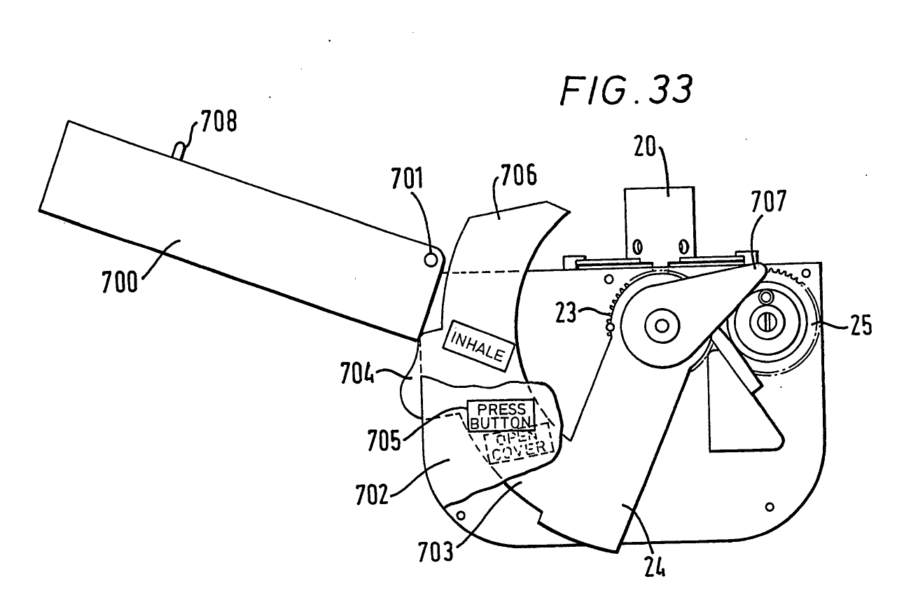

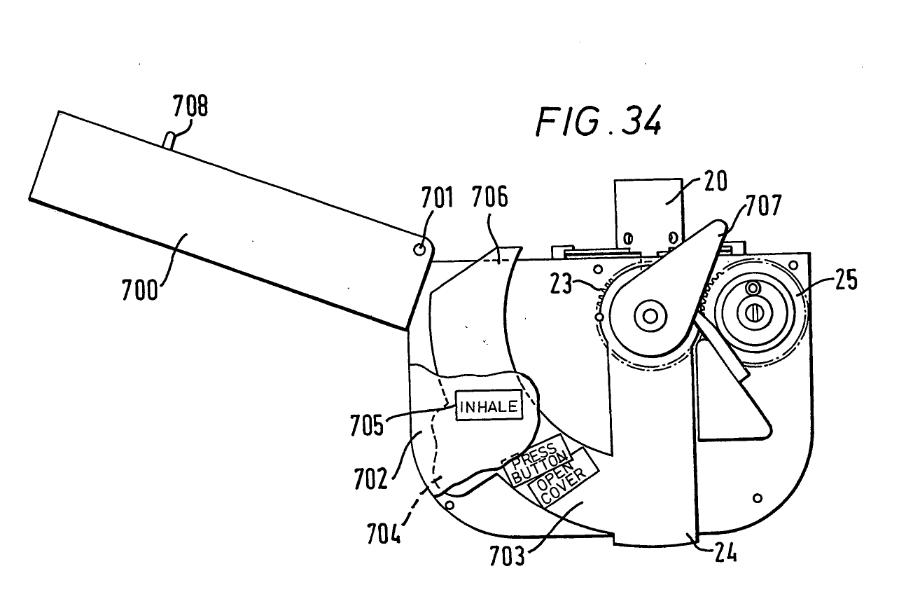

FIG. 32 shows the device in its rest position. The legend “OPEN COVER” is visible through the window 705. If a patient now opens the cover 700 this brings the device into the position shown in FIG. 33. It will be seen that the top rear edge of the cover has struck the cam 704 and moved the extension 703 through an angle such as to make the legend “PRESS BUTTON” visible through the window 705. If the user now presses the button 706 this causes the lever 24 to rotate, thus opening a powder-containing container, as described in connection with FIGS. 1 to 3. This brings the device into the position shown in FIG. 34, in which the legend “INHALE” is visible through the window 705. It will also be seen that in the position of FIG. 34 the tongue 707 protrudes upwardly. Accordingly, when the user, having inhaled, closes the cover, the tongue 707 is struck by a lug 708 on the underside of the cover, which pushes the lever 24, with its extension 703, back into the position shown in FIG. 32, once again causing the legend “OPEN COVER” to be displayed.

The device just described not only gives the step-by-step instructions to the user, thus reducing the risk of a patient being confused, but also makes it difficult for the patient to use the device other than in the intended manner, by virtue of the fact that the button 706, once depressed, is not again accessible until the user closes the cover and reopens it.

In the embodiments described above, reference is made to a mouthpiece. However, if the device was to be used for purposes other than oral inhalation some other outlet would be employed, e.g. a nosepiece.

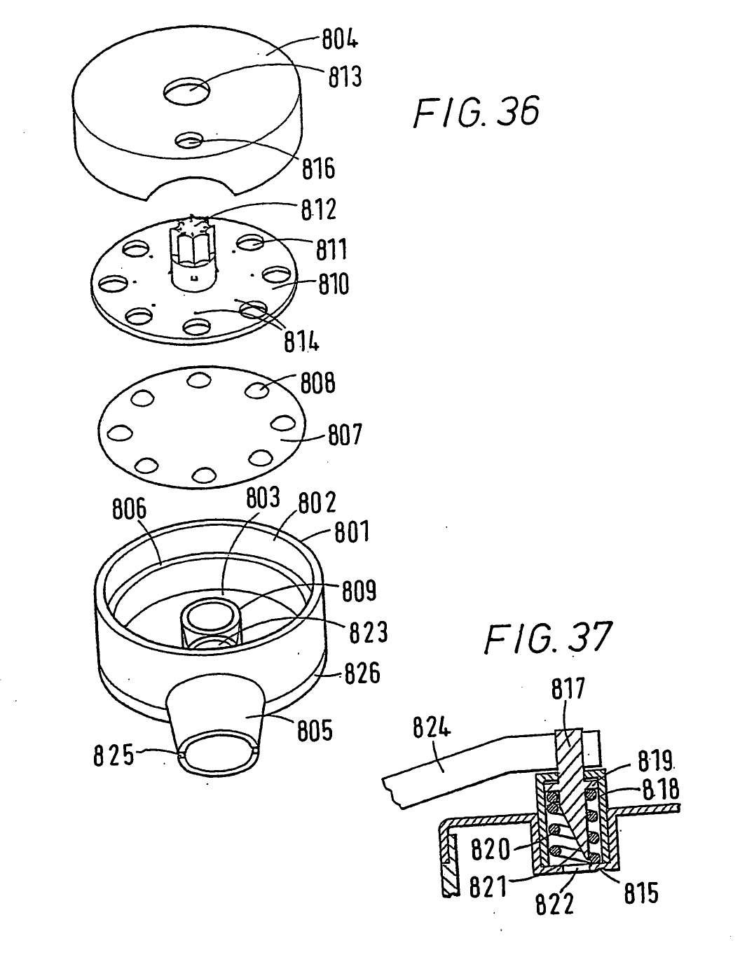

FIGS. 36 & 37 illustrate another embodiment of the invention. The device comprises a shallow cylindrical housing 801 of a plastic material which has a cylindrical chamber 802 therein. The chamber is closed at one end 803, herein considered the bottom of the chamber, and a removable cover 804 is a close fit over the chamber at the other end.

A mouthpiece outlet 805 projects outwardly from the cylindrical wall of the housing 801 and communicates with the interior of the chamber 802. A perforated guard, not shown, is provided in the mouthpiece to prevent any solid particles of an undesirably large size being inhaled by a patient inhaling through the mouthpiece.

A rim or shoulder 806 runs round the inside wall of the chamber 802 to provide an annular support on which a blister pack 807 may be located.

The blister pack 807 can conveniently be a foil laminate with a plurality of frangible containers or “blisters” 808 arranged in a circle. The blisters 808 are filled with medicament (e.g. having a formulation as described hereinbefore in any of the Examples 1-12) in particulate form, having a particle size in the range of 0.5-10 microns. The medicament may be with a pharmaceutically acceptable carrier such as lactose or starch in particulate form. The blister pack is of circular disc form, and is removably fitted inside the chamber so that it is replaceable when the individual doses of medicament contained in the blisters have been discharged.

The chamber 802 contains a central open cylindrical support column 809 upstanding from the bottom wall 803 of the chamber. A clamp disc member 810 is removably fitted inside the chamber 802 and has on its underside a plurality of locating pegs, not shown, which engage inside the support column. The clamp member 810 is rotatable inside the chamber. In use, the clamp member is placed on top of a blister pack 807 which has already been loaded into the chamber and is located on the support shoulder 806. The blister pack 807 is preferably a circular disc of foil laminate material with blisters or containers 808. The clamp member 810 has a plurality of apertures 811 or which are arranged in a circle and so spaced from each other that each of them will receive one of the blisters 808 of the blister pack 807. A knob 812 is upstanding from the clamp member 810 and when the lid 804 is fitted on the housing 801 the knob 812 will project through an aperture 813 in the top of the lid 804. This knob can be turned by the patient to rotate the clamp member 810 and since the blisters 808 of the blister pack 807 are located in the apertures 811 in the clamp plate 810 rotation of the clamp member will also rotate the blister pack. A plurality of protuberances or pips 814 are provided on the top of the clamp member 810 and engage in a recess 815, FIG. 37 on the underside of the cover 804 to make sure that the clamp plate is correctly aligned in position. As will be seen, the knob 812 is fluted to provide openings between the knob and the hole 813 through which air can enter the chamber 802 from the outside.

The cover 804 also has an aperture 816 in which a plunger 817 contained in a plunger housing 818 can be received. The plunger has an annular shoulder 819 and a spring 820 can bear between the shoulder 819 and the bottom of the plunger housing 818 to urge the plunger into an upper or inoperative position.

The plunger may be provided with a knife edge 821 or other means to enable the blister to be opened. When the plunger 817 is depressed against the action of the spring 820, the lower edge portion 821 of the plunger will pass through a blister 808 located in register with the plunger. Such engagement will open the blister, and permit the release of medicament therefrom. This action will so open the blister that when a patient inhales air will pass through the blister, the medicament being entrained in the air flow and exiting through the mouthpiece 805 via a transfer cavity 823 inside the chamber in communication with the mouthpiece 805. By rotation of the knob 812 the clamp member 810 and the blister pack 807 can be rotated to bring each blister in turn into location beneath the plunger. The various protuberances or pips 814 will in turn engage in the recess 815 to make sure that the blister pack is correctly registered with the plunger.

It is not essential that the plunger have a knife edge 821 to open the blister. If desired a needle can be used to perforate the blister or the plunger may have a pointed end or even a blunt end or another convenient opening means may be used.

The mouthpiece cover can have a locking member 824 which can be engaged with the plunger when the device is not in use to prevent accidental actuation of the plunger.

In use, the patient needing a dose of medicament may hold the device with the mouthpiece in his mouth. The patient then depresses the plunger to open the blister and give access to the medicament therefrom and inhales through the mouthpiece so that the medicament will be entrained in the airflow and will enter the lungs of the patient. If desired, the mouthpiece can be provided with air inlet apertures 825 to improve the airflow as the patient inhales.

In a modification not illustrated the underside of the blister pack can be supported on another clamp plate instead of the support rim or shoulder 806. The blister pack is conveniently arranged to provide a sufficient number of individual doses for a patient for use during a convenient period such as one day or more. The housing can be modified by providing an additional chamber, not visible, at the bottom, this additional chamber being closed by a removable cover 826. This additional chamber can be used to store replacement blister packs.

The mouthpiece may, if desired, be arranged so that a patient may use it to inhale through the nose.

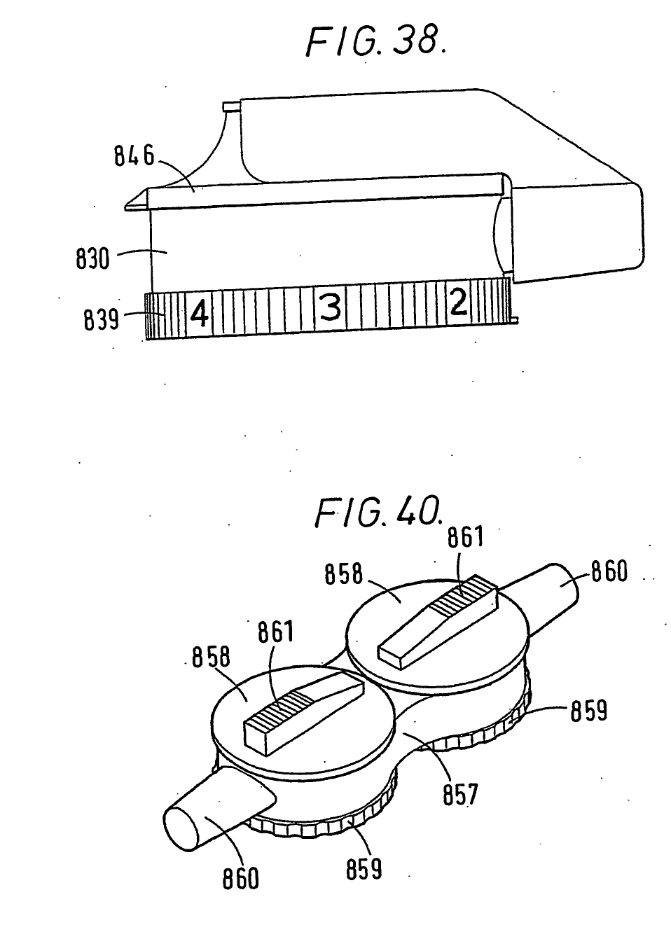

A modified device which does not use the clamp member 810 is illustrated in FIGS. 38 and 39. The device of this modification comprises a housing 830 having a chamber 831 therein. A mouthpiece 832 projects outwardly from the cylindrical wall of the housing 830 in a generally radical direction and communicates with the interior of the chamber 831. A perforated guard 833 is provided at the entrance to the mouthpiece 832. A rim or shoulder 834 runs round the inside wall of the chamber 831 to provide an annular support for a support member 835 in the form of a circular plate or disc. This support member is arranged to receive a blister pack 836. The blister pack 836 has a plurality of frangible containers 837 arranged in a circular row. These containers are in the form of “blisters” of a generally conical form as clearly shown in FIG. 39 and contain a medicament as described with reference to FIG. 36. The support member 835 has a plurality of holes 838 equal in number to the number of blisters 837 of the blister pack 836. The conical portion of one blister 837 is located in each of the holes 838 when the device is loaded and in use. An external rotatable member 839 with a knurled edge 840 is located in face contact with the bottom of the housing 830. A spindle or the like 841 with radial projections 842 extends centrally from the support member 835 through a hole 843 in the bottom of the housing 830 and into an opening 844 of complementary shape in a spigot 845 of the member 839. The spigot 845 passes through the hole 843 and the spindle 841 and 842 engages in the opening 844 so that rotation of the member 839 will cause similar rotation to the support member 835. A removable cover 846 fits on top of the housing 830. An opening 847 is provided in the cover 846 and engages a projection 848 in the housing 830 so as correctly to locate the cover. The cover 846 carries a bracket 849 on which a lever or trigger 850 is pivotally mounted. A plunger 851 is located on the lever or trigger 850 and extends through a hole 852 in the cover. A spring 853 is provided to bear between the trigger or lever 850 and the top of the cover 846 to urge the lever or trigger upwards.

The hole 852 is so positioned that each hole 838 in the support member 835 will register with this hole as the support member 835 is rotated.

When one of the holes 838 is in register with the hole 852 the trigger 850 can be depressed so that its plunger 851, which may be in the form of a needle, will pierce through the blister 837 located in that hole (i.e. pierce the top and the bottom of the blister) thereby permitting powder to exit from the blister. Some powder will fall into a tray-like compartment 854 inside the chamber 831. When the patient inhales, air passes through the pierced blister so that powder will be entrained in the airflow and will, with powder from the compartment 854, be withdrawn through the guard 833 and the mouthpiece 832. When the device is not in use, the mouthpiece 832 can be enclosed in a mouthpiece cover or sheath 855 which has a channel-like extension 856 which will engage with the bracket 849 to prevent the plunger 851 being depressed to enter through the hole 837. When the device is in use and the patient inhales through the mouthpiece 832 it is, of course, essential for air to be able to enter the interior of the chamber 831. Any suitable air inlets can be provided. Conveniently, however, air can enter through the hole 852 the plunger or needle 851 being smaller in diameter than the diameter of the hold 852 so that it serves as an air inlet.

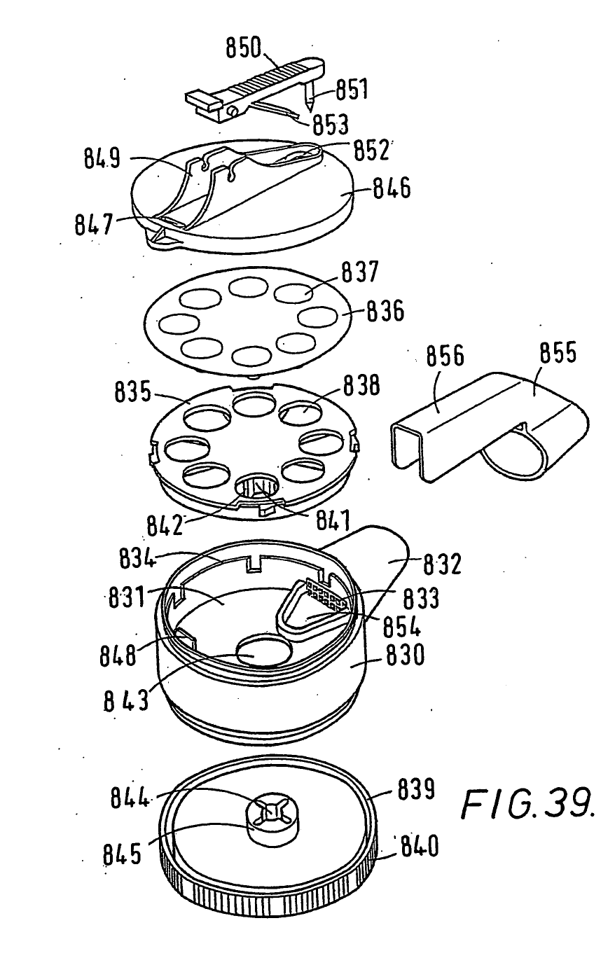

FIG. 40 illustrates a modified device which can conveniently be used to administer two different medicaments to a patient at separate times. Treatment of certain patients does require that they inhale two different kinds of medicament. In the device illustrated in FIG. 40, a common housing 857 contains two chambers equivalent to the chamber 802 of the embodiment illustrated in FIGS. 36 and 37 or to the chamber 831 of the embodiment illustrated in FIGS. 38 and 39. These two chambers are enclosed by removable covers 858 and blister packs contained in the chambers can be rotated in the manner previously described by rotation of knurled wheels, knobs or other members 859. Outlet mouthpieces 860 project outwardly from the common housing 857, each one of these outlets 860 leading into one of the chambers enclosed by the common housing. Trigger mechanisms 861 are provided to enable the blisters of the blister packs contained in the chambers to be pierced so that the contents thereof can be inhaled by the patient.

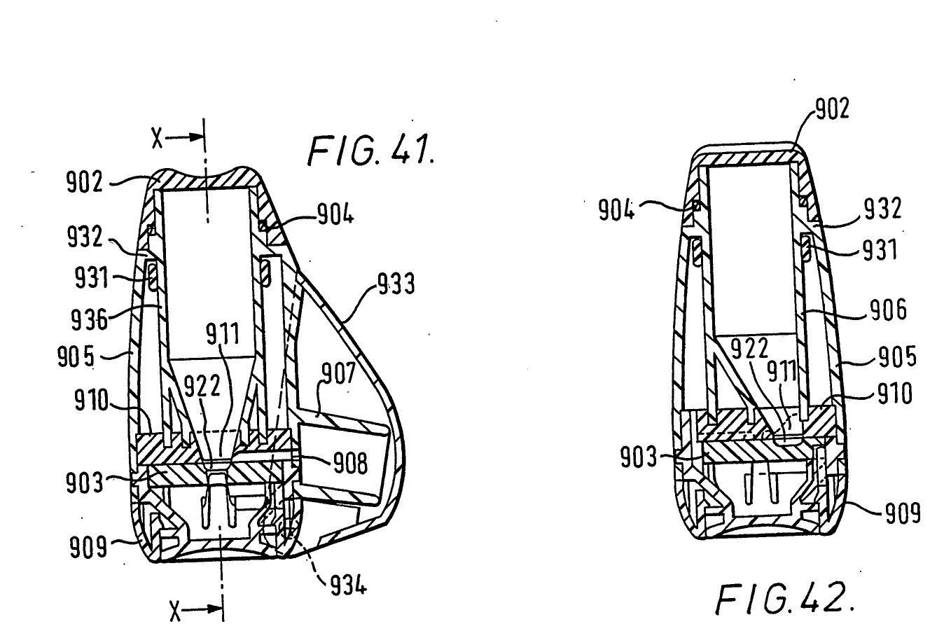

FIGS. 41 and 42 illustrate another embodiment of the present invention. The device shown in cross section in FIGS. 41 and 42 comprises a main body portion 905 which defines a reservoir 906 and a reservoir cover or end cap 902. The reservoir 906 contains a supply of medicament in powder form (e.g. having a formulation as described hereinbefore in any of the Examples 1-12). The reservoir cover 902 may be provided with a desiccant cartridge (not shown) to absorb moisture and reduce the risk of the powder in the reservoir absorbing moisture and undergoing agglomeration of the particles thereof. The cover 902 may be removably secured to the body 905 by any known means, for example by means of a screw thread or a snap fit, to enable refilling of the reservoir 906 with powder. Alternatively, the device may be intended to be disposable after exhaustion of the supply of medicament powder in the reservoir, in which case the cover 902 may be permanently secured to the body 905 by means of an interference fit or by use of an adhesive, ultrasonic welding or any other suitable method. A pharmaceutical grade rubber sealing ring 904 may be incorporated between the cover 902 and the body 905 to prevent ingression of moisture into the reservoir 906.

At its lower end the main body portion 905 is fitted with a base 910 which together with body 905 defines an aperture 911 which is offset from the vertical axis of the device and through which powder can pass from the reservoir to the dosing member 903. Powder is guided to the aperture by the walls of the reservoir which form a hopper. Extending laterally from the lower end of the main body 905 is mouthpiece 907. If, however, the device were intended for nasal inhalation this would be replaced by a nosepiece. Dosing member 903 having a metering recess 922 is mounted upon lower body portion 909 which is pivotally connected to main body 905 such that it may rotate about the vertical axis of the device. Lower body portion 909 serves to allow rotation of the dosing member 903 whilst maintaining the same axial alignment with base 910. It also urges the dosing member 903 into close contact with base 910. Dust cover 933 is attached to lower body portion 909 through pivot 934.

A weight 931 in the form of a ring encircles the reservoir 906 and is slidable longitudinally thereof. The locus of movement of the weight 931 is defined towards the top of the reservoir by an end stop 932 formed as an integral part of the body 905, and towards the bottom of the reservoir by base 910 which behaves as an anvil. It is understood that whilst the device described herein incorporates a weight for the purpose described below, the weight is not an essential element of the invention and it might be chosen to omit the incorporation of the weight.

The lower face of the base 910 is formed by a flat flexible rubber insert (not shown), while the upper face of the dosing member 903 is moulded with a flat contacting face to form a dynamic seal between the body and dosing member. These flat faces provide contacting surfaces between which there is substantially no clearance. Air and powder are thus excluded from the interface between the base 910 and dosing member 903 both in the static state and during the sliding motion of one face over the other minimising both loss of powder from and ingression of moisture to the reservoir 906 through the interface between the base 910 and dosing member 903. This type of dynamic or sliding seal obviates the need for any additional sealing means between base 910 and dosing member 903.

The contacting faces need not be provided with precision finishes to provide an effective seal. Any undulations in the flatness of the upper face of the dosing member will be compensated for by the flexible rubber insert to maintain an effective seal. Although adequate performance of the seal may be achieved using a rubber material having a hardness of below 80 Shore A, it has been found that optimal performance of the seal is achieved using a rubber material having a hardness of between 40 and 60 Shore A. If the hardness of the rubber is below 40 Shore A, the rubber insert tends to deform into metering recess 922, so scraping powder out of the recess and reducing the quantity of powder metered. On the other hand, if the hardness of the rubber is above 60 Shore A, the effectiveness of the seal may be impaired. Smooth finishes on both contacting faces are desirable to maintain a good seal, but good results have been obtained from contacting faces moulded directly from highly polished tooling with no additional manufacturing process.

The rubber insert may be made from butyl to provide the desired hardness and flexibility. However, butyl has a high coefficient of friction and tends to hinder movement of the contacting faces relative to each other. It is therefore preferable to use either chlorinated butyl or butyl laminated with a contacting face made of a layer of PTFE, polypropylene or polyethylene. Such rubber inserts may be manufactured by standard techniques and provide a contact face with reduced coefficient of friction. Alternatively, the contacting face may be subject to any other surface treatment that reduces friction, such as plasma modification or varnish.

PTFE is a particularly suitable material for this purpose due to its low coefficient of friction (below 0.1), though materials having coefficients of friction up to around 0.4 may be acceptable. Good results have been achieved using butyl laminated with a contacting face made of PTFE foil having a thickness of around 0.2 mm. The foil may be adhered to the rubber insert without glue using standard manufacturing techniques. If the PTFE foil is thinner than 0.2 mm, the foil tends to crumple during vulcanisation of the rubber, while if the foil is thicker than 0.2 mm, the insert becomes harder and the effectiveness of the seal may be impaired.

The contacting face of the dosing member may be integrally moulded with the dosing member of any suitable material, e.g. acetal resin. Alternatively, it will be understood that the contacting face of the dosing member may be formed by a flat flexible rubber insert as described above and the lower face of base 910 may be integrally moulded in one piece as part of base 910 from a suitable material. Alternatively, both faces may be formed by flat flexible rubber inserts as described.

In the embodiment described, the two faces are formed by the surfaces of flat discs. It will be appreciated that disc shapes are not essential. Contact faces may be formed by the surfaces of a frusto-cone and a correspondingly frusto-conical socket, by the contacting surfaces of two co-axial cylinder or by two correspondingly partially spherical contacting ball and socket surfaces.

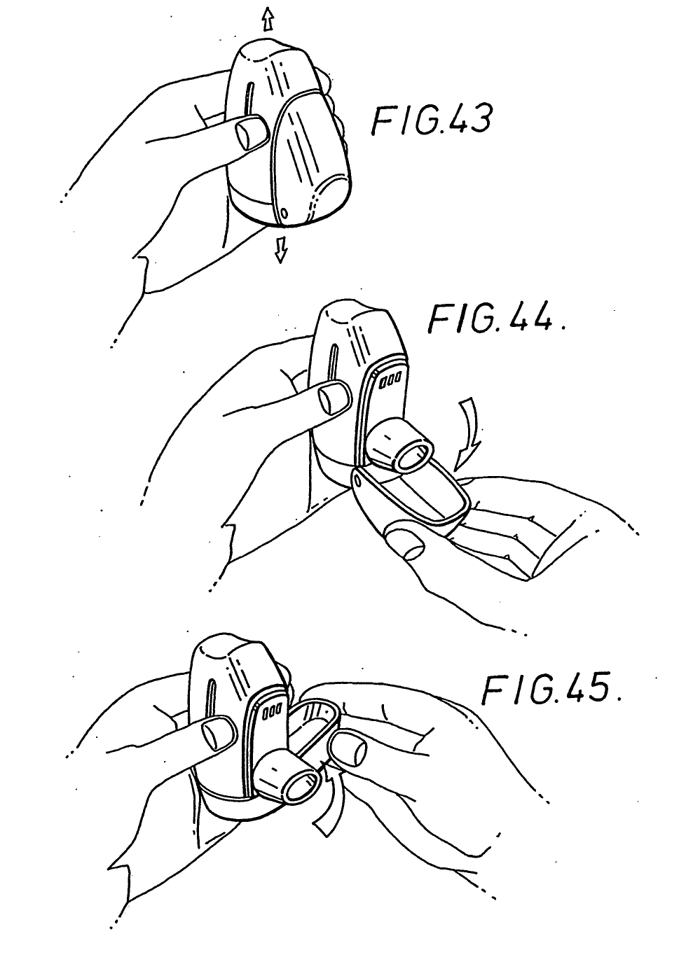

In operation, the user initially shakes the device in a generally upward and downward motion While maintaining the device in a generally upright orientation as shown in FIG. 43. Weight 931 is thereby caused to travel up and down the reservoir, so repeatedly striking end stop 932 and base 910. The jolts which this produces cause the powder in the reservoir to be urged downwardly and to enter the metering recess 922.

The user then opens dust cover 933, as shown in FIG. 44, and rotates the cover which is connected to lower body portion 909 as described above and shown in FIG. 45, to move the dust cover 933 away from the mouthpiece 907 to allow access thereto and to bring the recess 922 into alignment with the aperture 908 leading to the mouthpiece 907. The user knows when this position has been reached as the lower body portion 909 engages a stop (not shown) and will not move any further. The user then inhales through mouthpiece 907. After inhalation the user returns the lower body portion 909 to its initial position and closes the dust cover 933.

In the device shown in FIGS. 41 and 42 the aperture 911 is radially offset by an angle of 90° about the vertical axis of the device from the aperture 908 at the inner end of the mouthpiece to allow the dust cover and lower body portion 909 to be moved through 90° for ease of access to the mouthpiece. However, it will be appreciated that this angle can be substantially increased or slightly decreased according to the desired angle of rotation of the dust cover, lower body portion and dosing member.

It will be understood that the present disclosure is for the purpose of illustration only and the invention extends to modifications, variations and improvements thereto.

The application of which this description and claims form part may be used as a basis for priority in respect of any subsequent application. The claims of such subsequent application may be directed to any feature or combination of features described therein. They may take the form of product, method or use claims and may include, by way of example and without limitation, one or more of the following claims:

1. An inhalation device comprising plural doses of medicament in powder form, wherein said medicament is a pharmaceutical formulation comprising salmeterol or a pharmaceutically acceptable salt, solvate, or physiologically functional derivative thereof and an anticholinergic agent or a pharmaceutically acceptable salt, solvate, or physiologically functional derivative thereof, and a pharmaceutically acceptable carrier or excipient, and optionally one or more other therapeutic ingredients.

2. An inhalation device according to claim 1, wherein said pharmaceutical formulation comprises salmeterol xinafoate and said anticholinergic agent, and a pharmaceutically acceptable carrier or excipient, and optionally one or more other therapeutic ingredients.

3. An inhalation device according to claim 1, wherein the anticholinergic agent is selected from the group consisting of ipratropium bromide, oxitropium bromide, tiotropium bromide, atropine sulfate, revatropate and any mixtures thereof.

4. An inhalation device according to claim 3, wherein the anticholinergic agent is ipratropium bromide.

5. An inhalation device according to claim 3, wherein the anticholinergic agent is oxitropium bromide.

6. An inhalation device according to claim 3, wherein the anticholinergic agent is tiotropium bromide.

7. An inhalation device according to claim 1, wherein the pharmaceutically acceptable carrier or excipient is lactose.

8. An inhalation device according to claim 1, wherein the medicament is contained on a retainer comprising a mesh disc or velour disc.

9. An inhalation device according to claim 1, wherein the medicament is contained within a multi-dose blister pack.

10. An inhalation device according to claim 9, wherein at least one container for the medicament in said multi-dose blister pack is defined between two members peelably secured to one another, the device comprising means defining an opening station for the said at least one container; means for peeling the members apart at the opening station to open the container; and an outlet, communicating with the opened container, through which a user can inhale medicament in powder form from the opened container.

11. An inhalation device according to claim 10, adapted for use where the said two members are two sheets.

12. An inhalation device according to claim 11, adapted for use where the sheets are elongate sheets which define a plurality of medicament containers spaced along the length thereof, the device being provided with indexing means for indexing each container in turn in communication with the outlet.

13. An inhalation device according to claim 12, adapted for use where one of the sheets is a base sheet having a plurality of pockets therein, and the other of the sheets is a lid sheet, each pocket and the adjacent part of the lid sheet defining a respective one of the containers, the device comprising driving means for pulling the lid sheet and base sheet apart at the opening station.

14. An inhalation device according to claim 13, wherein the said driving means comprises lid driving means for pulling the lid sheet.

15. An inhalation device according to claim 14, comprising a rotatable index wheel having recesses therein, the wheel being engageable with the medicament pack so that the recesses each receive a respective pocket.

16. An inhalation device according to claim 15, wherein said index wheel and the lid driving means are interconnected so that the rotation of one correlates with the rotation of the other.

17. An inhalation device according to claim 16, wherein the index wheel and lid driving means are interconnected by a slipping clutch.

18. An inhalation device according to claim 17, wherein said slipping clutch comprises a first gear member which is movable with the index wheel and has a toothed surface, and a second toothed gear member which is movable with the lid driving means and has a toothed surface in meshing engagement with the toothed surface of the said first gear member, at least one of the toothed surfaces having a toothed portion which is movable back and forth with respect to the remainder of the toothed surface of which it is part.

19. An inhalation device according to claim 17, wherein the slipping clutch comprises first clutch means movable with the index wheel and second clutch means movable with the lid driving means, one of the clutch means comprising an annular array of serrations and the other of the clutch means comprising means which grippingly engage the serrations when less than a predetermined force is applied between the two clutch means and which slip with respect to the serrations when a force equal to or greater than the predetermined force is applied.

20. An inhalation device according to claim 14, wherein the lid driving means comprises a wheel on which the lid sheet is wound up, the said wheel having a winding surface which decreases in diameter when tension in the lid sheet increases.

21. An inhalation device according to claim 20, wherein the said wheel comprises a plurality of resiliently flexible arms each extending therefrom at an angle with respect to a radius.

22. An inhalation device according to claim 13, comprising indexing means engageable between adjacent pockets to cause each pocket in turn to be positioned in communication with the outlet.

23. An inhalation device according to claim 14, wherein the lid driving means comprises a pair of driving wheels which drivingly engage the lid sheet between them.

24. An inhalation device according to claim 23, wherein the said driving wheels are toothed wheels having interengaging teeth.

25. An inhalation device according to claim 13, comprising means for guiding the lid sheet and base sheet along separate paths at the opening station, the paths reuniting downstream of the opening station, the said driving means being located after the point where the paths reunite and being operable to drive both the lid sheet and base sheet.

26. An inhalation device according to claim 25, wherein the said driving means comprises a pair of toothed wheels having interengaging teeth.

27. An inhalation device according to claim 13, comprising at least one chamber for receiving the elongate medicament pack before opening, and for receiving the base sheet and lid sheet after peeling apart.

28. An inhalation device according to claim 27, wherein the elongate medicament pack and/or base sheet are held in coiled form by resilient coil-formers.

29. An inhalation device according to claim 1, operable in a plurality of steps, which comprises indicator means adapted to display to a user an instruction as to the next step once the preceding step has been taken.

30. An inhalation device according to claim 29, wherein the indicator means comprises an indicator member which carries a plurality of legends each constituting an instruction to the user, the indicator member being movable by a given step being carried out to display the legend relating to the next step.

31. An inhalation device according to claim 9, comprising a housing with a cylindrical chamber therein; an air inlet into said chamber; a support inside the chamber arranged to support said blister pack comprising a plurality of containers arranged in a circle; a plunger operable to engage a container registered therewith to open the container is such a way that air being inhaled by a patient will cause the medicament to be released therefrom; means for rotating the blister pack on the support to register each container in turn with the plunger; and, communicating with the interior of the chamber, a mouthpiece outlet through which a patient can inhale whereby medicament will be released from a container and entrained in the airflow produced by the patient so as to pass through the outlet.

32. An inhalation device according to claim 31, wherein said support is a rotatable plate with a plurality of holes therethrough, the holes being arranged in a circle and each being adapted to receive a container for medicament, a rotatable member being located outside the chamber and connected with the support so that rotation of the said member will cause rotation of the support; a mouthpiece outlet leading from the chamber in a substantially radial direction; and a perforated guard positioned so that air and medicament inhaled through the mouthpiece will first pass through said guard.

33. An inhalation device according to claim 31, wherein the support is a rim inside the chamber and a clamp member is fitted inside the chamber and on the support but is removable to permit the blister pack to be placed on the support and thereafter clamped between the clamp member and the support, the clamp member having a plurality of holes arranged in a circle to receive a plurality of containers, being rotatable and arranged to rotate with it the blister pack clamped between the clamp member and the support, wherein an external knob is provided to rotate the clamp member and an outlet mouthpiece leads substantially radially from the chamber.

34. An inhalation device according to claim 31, wherein the chamber has a cover which is removable to permit the blister pack to be inserted in the chamber and placed on the support, the plunger being carried by the cover.

35. An inhalation device according to claim 32, wherein the mouthpiece is enclosed in a removable mouthpiece cover, said mouthpiece cover having means for preventing operation of the plunger when the mouthpiece cover is fitted on the mouthpiece.

36. An inhalation device according to claim 31, wherein the blister pack is a circular disc having a plurality of frangible containers arranged in a circle and containing medicament in powder form.

37. An inhalation device according to claim 1, wherein the device comprises a body defining a reservoir for said medicament, an outlet through which a user can inhale, and a dosing member with at least one metering means formed therein, said dosing member being movable between a first position in which the at least one metering means communicates with said reservoir to receive a dose of medicament therefrom and a second position in which the at least one metering means communicates with said outlet to permit the user to inhale said dose.

38. An inhalation device according to claim 37, wherein the at least one metering means is formed in a face of the dosing member, the face being urged into contact against a similar mating face of the body at the lower end of the reservoir to form a dynamic seal.

39. An inhalation device according to claim 38 wherein the flexible material has a coefficient of friction of 0.4 or less.

40. An inhalation device according to claim 38, wherein the faces are flat.

41. An inhalation device according to claim 37, wherein the mating face of the body is made of a flexible material.

42. An inhalation device according to claim 41, wherein the mating face of the body comprises a rubber insert having a hardness of between 40 and 60 Shore A.

43. An inhalation device according to claim 42, wherein said rubber insert comprises chlorinated butyl or butyl laminated with a contacting face made of a layer of PTFE, polypropylene or polyethylene.

44. An inhalation device according to claim 37, wherein the face of the dosing member is of unitary construction with the dosing member.

45. An inhalation device according to claim 37, wherein said dosing member is rotatably movable with respect to said reservoir about a common central axis such that the metering means are selectively in communication with the reservoir to receive said dose of medicament therefrom or with the outlet to release the dose of medicament to the user.

46. An inhalation device according to claim 45, additionally comprising a reversibly removable reservoir cap to cover said reservoir and automatically load the metering means for use on removal of said cap.

47. An inhalation device according to claim 45, additionally comprising loading means for transferring medicament from the reservoir into the metering means.

48. An inhalation device according to claim 47, wherein said loading means comprises a resilient scraper means positioned to first push medicament powder into the metering means and then remove excess medicament powder from the face of the dosing member to ensure that each metering means contains a reproducible amount of medicament powder.

49. An inhalation device according to claim 37, wherein each metering means is in the form of a perforation or cavity for receipt of a pre-determined volume of medicament powder therein.

50. An inhalation device according to claim 37, wherein the metering member is a flat plate.

51. An inhalation device according to claim 37, additionally comprising counter means for providing a visual count of the number of doses of powdered medicament used or remaining within the reservoir.

52. An inhalation device according to claim 1, actuable in response to the breath of a user.

53. A method for the prophylaxis or treatment of a clinical condition in a mammal, such as a human, for which a selective β2-adrenoreceptor agonist and/or anticholinergic agent is indicated, which comprises:
providing an inhalation device of claim 1 comprising a therapeutically effective amount of a pharmaceutical formulation comprising salmeterol or a pharmaceutically acceptable salt, solvate, or physiologically functional derivative thereof and an anticholinergic agentor a pharmaceutically acceptable salt, solvate, or physiologically functional derivative thereof, and a pharmaceutically acceptable carrier or excipient, and optionally one or more other therapeutic ingredients, and
administering said pharmaceutical formulation to the mammal from said inhalation device.

54. A method according to claim 53, comprising administration of a therapeutically effective amount of a pharmaceutical formulation comprising salmeterol xinafoate and said anticholinergic agent, and a pharmaceutically acceptable carrier or excipient.

55. An inhalation device according to claim 53, wherein the anticholinergic agent is selected from the group consisting of ipratropium bromide, oxitropium bromide, tiotropium bromide, atropine sulfate, revatropate and any mixtures thereof.

56. An inhalation device according to claim 55, wherein the anticholinergic agent is ipratropium bromide.

57. An inhalation device according to claim 55, wherein the anticholinergic agent is oxitropium bromide.

58. An inhalation device according to claim 55, wherein the anticholinergic agent is tiotropium bromide.

59. A method according to claim 53, wherein the clinical condition is a disease associated with reversible airways obstruction such as asthma, chronic obstructive pulmonary disease (COPD), respiratory tract infection or upper respiratory tract disease.

60. A method according to claim 59, wherein the clinical condition is chronic obstructive pulmonary disease (COPD).
